(19) United States
(12) Patent Application Publication
(10) Pub. No.: US 2011/0245197 A1
(43) Pub. Date: Oct. 6, 2011

(54) HYPERSULFATED GLUCOPYRANOSIDES

(75) Inventor: Tahir AHMED, Coral Gables, FL (US)

(73) Assignee: OPKO Health, Inc., Miami, FL (US)

(21) Appl. No.: 13/074,549

(22) Filed: Mar. 29, 2011

Related U.S. Application Data
(60) Provisional application No. 61/319,687, filed on Mar. 31, 2010.

(51) Int. Cl. A61K 31/7024; C07H 11/04; C07H 11/00; A61P 29/00; A61P 11/06; A61P 37/08; A61P 31/06; A61P 11/00; A61P 19/02; A61P 17/00; A61P 1/00

(52) U.S. Cl. 514/53; 536/117; 536/118

(57) ABSTRACT

Hypersulfated disaccharides, preferably octasulfated sucrose, with utility in asthma or asthma related disorders are disclosed. The compounds may optionally be formulated with pharmaceutically acceptable excipients or delivery agents. The delivery agents are selected from the group consisting of natural or synthetic polymers, aerosols or other vehicles that facilitate the delivery or administration of the drug. The hypersulfated disaccharides are made from carbohydrate starting materials. Ion exchange or other suitable synthetic processes may be utilized to prepare the pharmaceuticals. The hypersulfated disaccharides are useful as anti-inflammatory agents.

This application claims priority to U.S. Application No. 61/319,687 filed Mar. 31, 2010 entitled “Hypersulfated Glucopyranosides”, herein incorporated by reference in its entirety.

### FIELD OF THE INVENTION

The present invention relates to pharmaceutical formulations comprising a hypersulfated glucopyranoside selected from, for example, β-(2S,3S,4S,5R)-fructofuranosyl-α-(1R,2R,3S,4S,5R)-glucopyranoside (sucrose) which is octasulfated and an optional additive selected from a pharmaceutically acceptable excipient or polymer or other vehicle depending upon the route of delivery. The formulations are useful in the treatment of a variety of inflammatory disorders and diseases in animals and people, and, in particular, pulmonary disorders selected from asthma and other conditions or diseases associated with inflammation of the lungs and airway.

### BACKGROUND OF THE INVENTION

U.S. Pat. No. 7,056,898 (the '898 patent) discloses and claims certain hypersulfated disaccharides and methods of using same to treat certain inflammatory disorders. This patent specifically describes the use of the claimed compounds to treat pulmonary inflammations including asthma and asthma-related pathologies, such as allergic reactions or an inflammatory disease or condition. The compounds disclosed therein are described as being capable of preventing, reversing and/or alleviating the symptoms of asthma and asthma-related pathologies, particularly the late phase response in asthma patients following antigen stimulation. U.S. Pat. No. 5,447,919 discloses the use of certain hypersulfated oligosaccharides to treat arteriosclerotic disorders. There is a need for an improved pulmonary or anti-inflammatory medication that can be delivered in small dosages to patients in need of treatment thereof on a convenient basis and which does not have the side effects associated with, for example, chronic administration of steroids or leukotriene receptor antagonists such as montelukast sodium.

The inventor has met this unmet need and has surprisingly found that certain octasulfated sucrose salts and formulations comprising such salts and an optional agent selected from the group consisting of a pharmaceutically acceptable natural or synthetic polymer as well as other vehicles that heretofore have been utilized to improve delivery of large compounds (e.g., those compounds having molecular weights of greater than 4,500 daltons as average molecular weight) have suitable absorption/bioavailability/efficacy and are effective as pharmaceutical formulations to treat patients having asthma related disorders as further recited herein. The octasulfated sucrose salts are also effective as inhalation agents.

### SUMMARY OF THE INVENTION

The present invention relates to pharmaceutical formulations comprising a compound of formula I and pharmaceutically acceptable salts thereof and an optional agent selected from the group consisting of a pharmaceutically acceptable excipient or synthetic polymer or natural polymer, or an oligomer or other agent. The compound in the formulation is a compound of formula I or a pharmaceutically acceptable salt thereof,

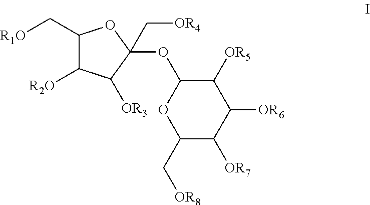

wherein R₁-R₈ are independently selected from the group consisting of H, SO₃H or PO₃H and provided that at least two of R₁-R₈ is selected from SO₃H or PO₃H. The present invention also relates to formulations having a compound of formula I wherein at least three of R₁-R₈ are selected from SO₃H or PO₃H. The present invention further relates to formulations having compounds of formula I wherein at least four of R₁-R₈ are selected from SO₃H or PO₃H. The present invention further relates to formulations having compounds of formula I wherein at least five of R₁-R₈ are selected from SO₃H or PO₃H. The present invention preferably relates to a compound of formula I and pharmaceutically acceptable salts thereof wherein R₁-R₈ are selected from SO₃H. The present invention also relates to formulations having a compound of formula I wherein R₁-R₈ are independently selected from SO₃H or PO_{S}H. The invention further includes pro-drugs, derivatives, active metabolites, partially ionized and fully ionized derivatives of the compounds of formula I and stereoisomers thereof. The monomers which make up the disaccharides of the invention may be D or L isomers and the hydroxyl moieties or sulfated or phosphated versions thereof around the carbocyclic ring (or acyclic versions or intermediates thereof) may have the alpha or beta designation at any particular stereocenter. The linking oxygen atom between the monosaccharide moieties may also be alpha or beta. The molecular weight of the compounds of the invention is typically less than 1,000 daltons.

The present invention also relates to a pharmaceutical formulation comprising

(i) a compound of formula I and pharmaceutically acceptable salts thereof.

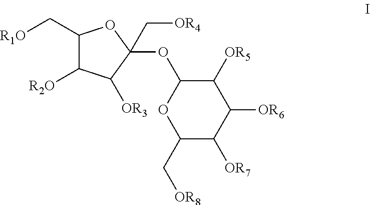

wherein R₁-R₈ are independently selected from H, SO₃H or POSH and, optionally,
(ii) a pharmaceutically acceptable excipient and provided that at least two of R₁-R₈ are selected from SO₃H or PO_{S}H.

The present invention also relates to a pharmaceutical formulation comprising

(i) a compound of formula I and pharmaceutically acceptable salts thereof wherein R₁-R₈ are independently selected from SO₃H or PO₃H and, optionally,
(ii) an additive selected from the group consisting of a pharmaceutically acceptable natural or synthetic polymer or a pharmaceutically acceptable excipient.

The invention relates to a pharmaceutical formulation comprising

(i) a compound of formula I and pharmaceutically acceptable salts thereof wherein R₁-R₈ are selected from SO₃H and
(ii) an additive selected from the group consisting of a pharmaceutically acceptable natural or synthetic polymer or a pharmaceutically acceptable excipient.

In another embodiment, the present invention relates to a pharmaceutical formulation comprising (i) a compound of formula II

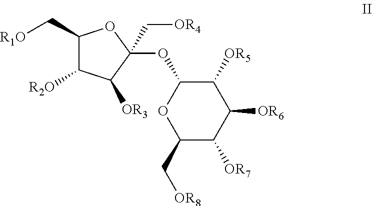

and pharmaceutically acceptable salts thereof wherein R₁-R₈ are independently selected from the group consisting of H, SO₃H or PO₃H and, optionally (ii) an additive selected from the group consisting of a pharmaceutically acceptable excipient or natural or synthetic polymer and wherein at least two of R₁-R₈ are selected from SO₃H or PO₃H. The fully ionized sodium salt of the octasulfated sucrose of formula I is designated as Compound 1a.

In a preferred embodiment, the invention relates to a pharmaceutical formulation comprising (i) a compound of formula II and pharmaceutically acceptable salts thereof wherein R₁-R₈ is SO₃H and, optionally, (ii) an additive selected from the group consisting of a pharmaceutically acceptable excipient or natural or synthetic polymer.

The present invention also relates to oral dosage forms comprising a compound of formula I or II wherein R₁-R₈ have any of the designations shown above and their pharmaceutically acceptable salts and an additive selected from the group consisting of a pharmaceutically acceptable excipient or natural or synthetic polymer.

The present invention also relates to inhalation dosage forms comprising a compound of formula I or II wherein R₁-R₈ have any of the designations shown above and their pharmaceutically acceptable salts and a pharmaceutically acceptable additive that is suitable for or to assist delivery by inhalation means.

The present invention also encompasses a method of treating an inflammatory condition in a mammal in need of treatment thereof comprising administering a pharmaceutically effective amount of a formulation comprising a compound of formula I and pharmaceutically acceptable salts thereof wherein R₁-R₈ are independently selected from SO₃H, PO₃H or H and provided that at least two of R₁-R₈ is SO₃H or PO₃H and, optionally, a pharmaceutically acceptable excipient or agent selected from the group consisting of a pharmaceutically acceptable natural or synthetic polymer or oligomer or agent that facilitates the delivery of a compound of formula I or II into the bloodstream and/or to a target site.

### BRIEF DESCRIPTION OF THE DRAWINGS

The invention will be described in the following drawings.

FIG. 1A shows a graph comparing the percentage change in specific pulmonary airflow resistance (measured as cm H₂O/L/sec) (i.e., the SR_{L}) following the indicated time after antigen administration (time=0) of sheep's responses to exposure to antigen only (closed circles) (control) and antigen plus an oral dosage of 25 mg×1 (25 mg/40 kg) of the fully ionized sodium salt octasulfated sucrose (aka Compound 1a) in a Carbopol/lactose formulation designated as GS-RD1-3 (open triangles). The GS-RD1-3 formulation having Compound 1a was administered ninety minutes before antigen challenge (i.e., −1.5 hr). Data are shown as antigen-induced mean plus or minus SE % change in SR_{L} in five sheep (n=5) exposed to antigen first with no drug and then again several weeks later with antigen plus GS-RD1-3 (compound 1a).

FIG. 1B shows a bar graph illustrating the effect of pretreatment on airway hyperresponsiveness (AHR) in allergic sheep. Data are shown as mean plus or minus SE PD₄ (airway responsiveness) in breath units at baseline and at 24 hours post-antigen challenge in a group of sheep (n=5) exposed to antigen first with no drug and then again with antigen several weeks later following pretreatment (90 minutes beforehand) with an oral dose of GS-RD1-3 (Compound 1a) (25 mg/40 kg) in oral capsule form (25 mg×1) single dose. PD₄₀₀ is defined as the provocating dose of carbochol in breath units which caused a 400% increase in SR_{L}. One breath unit is one breath of 1% solution of carbochol. PD₄₀₀ is an indicator of airway responsiveness.

FIG. 2A shows a graph comparing the percentage change in specific pulmonary airflow resistance (i.e., the SR_{L}) following the indicated time after antigen administration (time=0) of sheep's responses to exposure to antigen only (closed circles) (control) and antigen plus an oral dosage of 25 mg×2 (50 mg/40 kg) of the octasulfated sucrose Carbopol/lactose formulation designated as GS-RD1-3 or the fully ionized sodium salt form of octasulfated sucrose (Compound 1a) (open triangles). Data are shown as antigen-induced mean plus or minus SE % change in SR_{L} in five sheep (n=5) exposed to antigen first with no drug and then again several weeks later with antigen plus GS-RD1-3. GS-RD1-3 was orally administered in capsule form 1.5 hours before antigen exposure.

FIG. 2B shows a bar graph illustrating the effect of pretreatment on airway hyperresponsiveness (AHR) in allergic sheep. Data are shown as mean plus or minus SE PD₄₀₀ (airway responsiveness) in breath units at baseline and at 24 hours post-antigen challenge in a group of sheep (n=5) exposed to antigen first with no drug and then again with antigen several weeks later following pretreatment (1.5 hours) with an oral dose in capsule form of GS-RD1-3 of 25 mg×2 (50 mg/40 kg).

FIG. 3A shows a graph comparing the percentage change in specific pulmonary airflow resistance (measured as cm H₂O/L/sec) (i.e., the SR_{L}) following the indicated time after antigen administration (time=0) of sheep's responses to exposure to antigen only (closed circles) (control) and antigen plus an oral dosage of 25 mg capsules×2 of the octasulfated sucrose formulation designated as GS-RD1-2 (no Carbopol) or the fully ionized sodium salt form of octasulfated sucrose (aka Compound 1a) (open triangles). Data are shown as antigen-induced mean plus or minus SE % change in SR_{L} in five sheep (n=5) exposed to antigen first with no drug and then again several weeks later with antigen after being pretreated (90 minutes before antigen exposure) with a total of 2×25 mg capsules of GS-RD1-2.

FIG. 3B shows a bar graph illustrating the effect of pretreatment on airway hyperresponsiveness (AHR) in allergic sheep. Data are shown as mean plus or minus SE Pam (airway responsiveness) in breath units at baseline and at 24 hours post-antigen challenge in a group of sheep (n=5) exposed to antigen first with no drug and then again with antigen several weeks later following pretreatment (90 minutes before exposure) with an oral dose of GS-RD1-2 (Compound 1a) of 25 mg×2 (50 mg/40 kg).

FIG. 4A shows a graph comparing the percentage change in specific pulmonary airflow resistance (measured as cm H₂O/L/sec) (i.e., the SR_{L}) following the indicated time after antigen administration (time=0) of sheep's responses to exposure to antigen only (closed circles) (control) and antigen plus an oral dosage of one 25 mg capsule/day given in the evening for three days before antigen exposure (25 mg/40 kg/day) of the octasulfated sucrose formulation designated as GS-RD1-3 (Compound 1a) (open triangles). Data are shown as antigen-induced mean plus or minus SE % change in SR_{L} in five sheep (n=5) exposed to antigen first with no drug and then again several weeks later with antigen after being pretreated for three days (×3 days) before antigen exposure with one single 25 mg capsule dose of the formulation designated as GS-RD1-3 administered in the evening (P.M. dose). Antigen challenge was 15 hours after the last 25 mg dose.

FIG. 4B shows a bar graph illustrating the effect of pretreatment on airway hyperresponsiveness (AHR) in allergic sheep. Data are shown as mean plus or minus SE PD₄₀₀ (airway responsiveness) in breath units at baseline and at 24 hours post-antigen challenge in a group of sheep (n=5) exposed to antigen first with no drug and then again several weeks later following pretreatment for three days before antigen exposure with an oral dose of GS-RD1-3 (Compound 1a) (25 mg/40 kg) administered in the evening for three days (25 mgs/40 kg/day) in the form of one 25 mg capsule per day. Antigen challenge was 15 hours after the last dose.

FIG. 5A shows a graph comparing the percentage change in specific pulmonary airflow resistance (measured as cm H₂O/L/sec) (i.e., the SR_{L}) following the indicated time after antigen administration (time=0) of sheep's responses (n=5) to exposure to antigen only (closed circles) (control) and antigen plus an oral dosage of one 25 mg (25 mg/40 kg) of the octasulfated sucrose formulation (absent Carbopol) designated as GS-RD1-2 (Compound 1a) given for three days in the evening before antigen exposure (open triangles). Data are shown as antigen-induced mean plus or minus SE % change in SR_{L} in five sheep (n=5) exposed to antigen first with no drug and then again several weeks later with antigen after being pretreated for three days (×3 days) before antigen exposure with one 25 mg capsule dose of GS-RD1-2 per day administered in the evening (P.M. dose). Antigen challenge was 15 hours following the last 25 mg capsule administration.

FIG. 5B shows a bar graph illustrating the effect of pretreatment on airway hyperresponsiveness (AHR) in allergic sheep. Data are shown as mean plus or minus SE PD₄₀₀ (airway responsiveness) in breath units at baseline and at 24 hours post-antigen challenge in a group of sheep (n=5) exposed to antigen first with no drug and then again with antigen several weeks later following pretreatment for three days before exposure with an oral dose of GS-RD1-2 (Compound 1a) (25 mg/40 kg) administered in the evening on three successive days as one 25 mg capsule. Antigen exposure occurred 15 hours after the last 25 mg capsule treatment.

FIG. 6A shows a graph comparing the percentage change in specific pulmonary airflow resistance (measured as cm H₂O/L/sec) (i.e., the SR_{L}) following the indicated time after antigen administration (time=0) of sheep's responses to exposure to antigen only (closed circles) (control) and antigen plus two daily oral capsule dosage form having 25 mg of the octasulfated sucrose Compound 1a and 50 mg of an additive selected from Carbopol 934 P NF(open triangles) (1:2 wt/wt ratio api/additive) and lactose filler with the formulation designated as GS-RD1-3. Data are shown as antigen-induced mean plus or minus SE % change in SR_{L} in five sheep (n=5) exposed to antigen first with no drug and then again several weeks later with antigen after being pretreated for three days (×3 days) before antigen exposure with two doses of 25 mgs of Compound 1a/50 mgs Carbopol 934P NF/lactose filler administered in the evening (P.M. dose) in capsule form. Antigen challenge occurred 15 hours following the last 2×25 mg Compound 1a treatment.

FIG. 6B shows a bar graph illustrating the effect of pretreatment on airway hyperresponsiveness (AHR) in allergic sheep. Data are shown as mean plus or minus SE PD₄₀₀ (airway responsiveness) in breath units at baseline and at 24 hours post-antigen challenge in a group of sheep (n=5) exposed to antigen first with no drug and then again with antigen several weeks later following pretreatment for 3 days before exposure with two daily oral capsule doses of a formulation comprising Compound 1a (25 mgs) and Carbopol 934P (50 mgs) and lactose filler administered in the evening in capsule form (25 mgs×2) (formulation GS-RD1-3). Antigen challenge occurred 15 hours following the last 2×25 mg Compound 1a treatment.

FIG. 7A shows a graph comparing the percentage change in specific pulmonary airflow resistance (measured as cm H₂O/L/sec) (i.e., the SR_{L}) following the indicated time after antigen administration (time=0) of sheep's responses to exposure to antigen only (closed circles) (control) and antigen plus two daily oral capsule dosage forms having 25 mg of the octasulfated sucrose Compound 1a (open triangles) with the formulation designated as GS-RD1-2 (absent Carbopol). Data are shown as antigen-induced mean plus or minus SE % change in SR_{L} in five sheep (n=5) exposed to antigen first with no drug and then again several weeks later with antigen after being pretreated for three days (×3 days) before antigen exposure with a daily dosage of 50 mgs of Compound 1a administered in the evening (P.M. dose) in capsule form (2 capsules per day administered at the same time or immediately following one another). Antigen exposure occurred 15 hours after the last 2×25 mg dosing.

FIG. 7B shows a bar graph illustrating the effect of pretreatment on airway hyperresponsiveness (AHR) in allergic sheep. Data are shown as mean plus or minus SE PD₄₀₀ (airwayresponsiveness) in breath units at baseline and at 24 hours post-antigen challenge in a group of sheep (n=5) exposed to antigen first with no drug and then again with antigen several weeks later following pretreatment for 3 days before exposure with a daily oral dose of a formulation comprising Compound 1a (25 mgs) administered in the evening in capsule form (formulation GS-RD1-2) as two 25 mg capsules/day to provide a total of 50 mgs/day of active ingredient each day for the three day period. Antigen exposure occurred 15 hours after the last 50 mg treatment.

FIG. 8A shows a graph comparing the percentage change in specific pulmonary airflow resistance (measured as cm H₂O/L/sec) (i.e., the SR_{L}) following the indicated time after antigen administration (time=0) of sheep's responses (n=4) to exposure to antigen only (closed circles) (control) and antigen plus two daily oral dosage capsule forms each having sucrose (25 mgs) and 50 mg of an additive selected from Carbopol 934 P (open triangles) and lactose filler. Data are shown as antigen-induced mean plus or minus SE % change in SR_{L} in four sheep (n=4) exposed to antigen first with no drug and then again several weeks later with antigen after being pretreated for three days (×3 days) before antigen exposure with a daily dosage of the 25 mg sucrose/Carbopol 934P(50 mg)/lactose formulation administered in the evening (P.M. dose) in capsule form (2 capsules per day). Antigen exposure occurred 15 hours following the last evening dose.

FIG. 8B shows a bar graph illustrating the effect of pretreatment on airway hyperresponsiveness (AHR) in allergic sheep. Data are shown as mean plus or minus SE PD₄₀₀ (airway responsiveness) in breath units at baseline and at 24 hours post-antigen challenge in a group of sheep (n=4) exposed to antigen first with no drug and then again with antigen several weeks later following pretreatment for 3 days before exposure with a daily oral dose of a formulation comprising sucrose (25 mgs), Carbopol 934 P(50 mgs) and lactose filler administered in the evening in capsule form as two capsules/day. Antigen exposure occurred 15 hours following the last evening dose of the sucrose/Carbopol/lactose formulation.

FIG. 9A shows a graph comparing the percentage change in specific pulmonary airflow resistance (measured as cm H₂O/L/sec) (i.e., the SR_{L}) following the indicated time after antigen administration (time=0) of sheep's responses (n=2) to exposure to antigen only (closed circles) (control) and antigen plus an inhaled dosage form having 5 mg of Compound 1a (open circles) with the formulation designated as MD-1688-76. Data are shown as antigen-induced mean plus or minus SE % change in SR_{L} in two sheep (n=2) exposed to antigen first with no drug and then again several weeks later with antigen after being pretreated (30 minutes before antigen exposure) with the 5 mg inhalation dose.

FIG. 9B shows a bar graph illustrating the effect of pretreatment on airway hyperresponsiveness (AHR) in allergic sheep. Data are shown as mean plus or minus SE PD₄₀₀ (airway responsiveness) in breath units at baseline and at 24 hours post-antigen challenge in a group of sheep (n=2) exposed to antigen first with no drug and then again with antigen several days later following pretreatment (30 minutes before exposure) with the inhalation formulation designated as MD-1688-76 (having 5 mgs of Compound 1a).

FIG. 10A shows a graph comparing the percentage change in specific pulmonary airflow resistance (measured as cm H₂O/L/sec) (i.e., the SR_{L}) following the indicated time after antigen administration (time=0) of sheep's responses (n=5) to exposure to antigen only (closed circles) (control) and antigen plus a single daily inhalation dose administered thirty minutes before exposure having 10 mgs of Compound 1a (designated as formulation MD-1688-76). Data are shown as antigen-induced mean plus or minus SE % change in SR_{L} in five sheep (n=5) exposed to antigen first with no drug and then again several weeks later with antigen after being pretreated (thirty minutes before antigen exposure) with an inhalation dose of 10 mgs of compound 1a.

FIG. 10B shows a bar graph illustrating the effect of pretreatment on airway hyperresponsiveness (AHR) in allergic sheep. Data are shown as mean plus or minus SE PD₄₀₀ (airway responsiveness) in breath units at baseline and at 24 hours post-antigen challenge in a group of sheep (n=5) exposed to antigen first with no drug and then again with antigen several weeks later following pretreatment (thirty minutes before antigen exposure) with an inhalation dose of 10 mgs Compound 1a (designated as formulation MD-1688-76).

FIG. 11 shows a graph comparing the percentage change in specific pulmonary airflow resistance (measured as cm H₂O/L/sec) (i.e., the SR_{L}) following the indicated time after antigen administration (time=0) of sheep's responses (n=5) to exposure to antigen only (closed circles) (control) and antigen plus an aerosol formulation having 0.5 mg/kg of the aluminum salt of octasulfated sucrose (approximately 20 mgs) (open circles). Data are shown as antigen-induced mean plus or minus SE % change in SR_{L} in five sheep (n=5) exposed to antigen first with no drug and then again several weeks later with antigen after being pretreated (thirty minutes before antigen exposure) with an inhalation dosage of 20 mgs of the aluminum salt octasulfated sucrose aerosol formulation. The average sheep weight was 40 kg.

### DETAILED DESCRIPTION

The present invention relates to pharmaceutical formulations and uses thereof wherein the formulation comprises a compound of formula I and pharmaceutically acceptable salts thereof

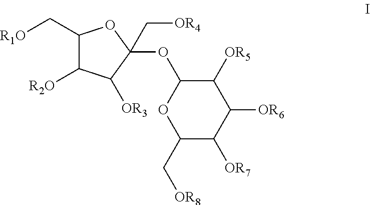

wherein R₁-R₈ are independently selected from the group consisting of H, SO₃H or PO₃H and provided that at least two of R₁-R₈ is selected from SO₃H or PO₃H and, optionally, a delivery agent selected from the group consisting of a pharmaceutically acceptable natural or synthetic polymer, oligomer or agent that facilitates the delivery of compound I into the bloodstream. Pharmaceutically acceptable excipients are also suitable as excipients that can be combined with the active ingredient of formula I. The term “pharmaceutically acceptable natural or synthetic polymer” generally means a pharmaceutically acceptable naturally derived or synthetic polymer having repeating units of a monomer or monomeric unit having a carbon chain or backbone (saturated or unsaturated or having both unsaturated and saturated monomers) with side chain substituents on the monomeric unit(s). Such polymers can be homopolymers or copolymers of repeating monomeric units wherein adjacent monomers can be the same or different. The side chain substituents include carboxylic acid groups or other polar groups selected from hydroxyl or amino groups and which can be further substituted with, for example, sulfate or phosphate groups. The polymers can be crosslinked. The preferred monomer is an acrylic acid residue and which forms carbomers. The molecular weight of such polymers can be around 500,000 to about 4 Billion. The molecular weight between crosslinks (M_{C}) can be, for example, for Carbopol 941, an estimated 104,400 g/mole. Additional polymers and drug delivery enhancing agents are described subsequently in the specification.

The present invention also relates to a pharmaceutical formulation comprising

(i) a compound of formula I and pharmaceutically acceptable salts thereof wherein R₁-R₈ are independently selected from SO₃H or PO_{S}H and, optionally,
(ii) an additive selected from the group consisting of a pharmaceutically acceptable excipient or natural or synthetic polymer.

In another embodiment, the present invention relates to a pharmaceutical formulation comprising (i) a compound of formula II

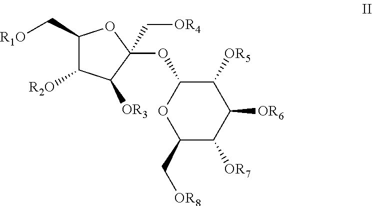

and pharmaceutically acceptable salts thereof wherein R₁-R₈ are independently selected from the group consisting of H, SO₃H or PO₃H and, optionally,
(ii) an additive selected from the group consisting of a pharmaceutically acceptable excipient or natural or synthetic polymer and wherein at least two of R₁-R₈ are selected from SO₃H or PO₃H.

In a preferred embodiment, the invention relates to a pharmaceutical formulation comprising (i) a compound of formula II and pharmaceutically acceptable salts thereof

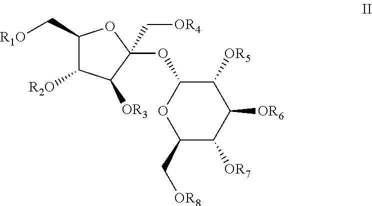

wherein R₁-R₈ is selected from SO₃H and, optionally,
(ii) an additive selected from the group consisting of a pharmaceutically acceptable excipient or natural or synthetic polymer.

The present invention also relates to oral dosage forms comprising a compound of formula I or II and their pharmaceutically acceptable salts with R₁-R₈ as defined above and an additive selected from the group consisting of a pharmaceutically acceptable excipient or natural or synthetic polymer.

The present invention also relates to inhalation formulations including but not limited to aerosol formulations comprising a compound of formula I or II and their pharmaceutically acceptable salts with R₁-R₈ as defined above and an additive selected from the group consisting of a pharmaceutically acceptable excipient and which is suitable for delivery by inhalation means.

The present invention also encompasses a method of treating or alleviating an inflammatory condition comprising administration of (i) a pharmaceutically effective amount of a formulation comprising a compound of formula I

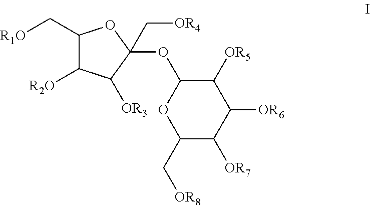

and pharmaceutically acceptable salts thereof wherein R₁-R₈ are independently selected from SO₃H, PO₃H or H and provided that at least two of R₁-R₈ is SO₃H or PO₃H and, optionally,
(ii) an additive selected from the group consisting of a pharmaceutically acceptable excipient or natural or synthetic polymer.

The present invention preferably relates to a pharmaceutical formulation comprising a compound of formula I or II wherein R₁-R₈ are selected from SO₃H and their pharmaceutically acceptable salts and, optionally, an additive selected from the group consisting of a pharmaceutically acceptable excipients or a natural or synthetic polymer.

In a preferred embodiment, the compounds in the formulation are selected from a metal salt of a compound of formula I or II wherein each sulfate group around the disaccharide is ionized to form a metal salt wherein the metals are selected from, for example, sodium. In addition, other salts including ammonium or amine salts may form at the sulfate positions. The most preferred compound is Compound 1a which is the fully ionized sodium salt form of octasulfated sucrose. The fully ionized aluminum salt is not effective.

The compounds can generally be prepared by a process which comprises treating the corresponding tri-, tetra-, penta- or hexaxaccharide with a sulfating agent and subsequently converting the reaction product into a salt form. Sucrose, raffinose, melezitose and stachyose are examples of saccharides that are utilized to form compounds of the invention. The sulfating agents are selected from those known to skill in the art and include, for example, SO₃-pyridine, SO₃-trimethylamine, SO₃-dioxan and SO₃-dimethylformamide. Chlorosulfonic acid and sulfuric acid and piperidine N-sulfate may also be used. Ion exchange may also be used to form, for example, the sodium salt of octasulfated sucrose which can be formed by treating the aluminum salt with an ion exchange resin to form, for example, the sodium salt. In general, sucrose may be treated with pyridine sulfur trioxide in anhydrous pyridine/DMF under warm conditions with stirring. After 6 to 18 hours at 55 to 65° C., the reaction mixture is cooled to room temperature and worked up to form a solid residue. This residue is re-dissolved in water while adjusting the pH to around 6.8 with sodium hydroxide solution to form, after treatment with activated charcoal and filtration, supersulfated material as a white solid. This material can then be run through a size exclusion chromatography column and eluted with ammonium bicarbonate to form the ammonium salt of the supersulfated sucrose. This can then be passed through a suitable ion exchange column (e.g. sodium or other cation of choice) to form a suitable salt of the hypersulfated sucrose.

Without being limited herein, it is understood that carbohydrates or complex carbohydrates are chiral molecules with hydroxyl groups as well as sulfate groups or phosphate groups present on the ring with set or absolute stereochemistry.

It is generally understood that the source of the polysaccharide which generates the oligosaccharides and disaccharides utilized in the formulations of the invention will determine, for the most part, the absolute stereochemistry of the chiral centers around the carbohydrate rings. Additional sulfate groups are added by chemical means by the process described generally above or by any known means to afford the most active moieties (hypersulfated disaccharides and salts thereof) which are further purified to form pharmaceutical grade disaccharides which are further formulated with an additive and processed into a dosage form suitable for administration to a mammal or other organism in need of treatment thereof.

Nuclear magnetic resonance imaging and/or other known structure identification methods may be used to determine the chemical structures of the molecules obtained from depolymerizing heparin (derived from any known source thereof) or other selected polysaccharide. In the event the compounds are made synthetically or semi-synthetically, the skilled artisan can use standard organic chemistry techniques to protect the desired hydroxyl moiety with a protecting group known to those of ordinary skill in the art.

A compound of formula I or II as described above (or mixtures thereof) is then formulated with an additive to form the formulations of the invention. The additive is selected from the group consisting of a pharmaceutically acceptable excipient or any natural or synthetic polymer (as further described below). The term “polymer” means a pharmaceutically acceptable natural or synthetic polymer. The term “pharmaceutically acceptable natural or synthetic polymer” means that the polymer is safe as administered to animals, including humans, in an administered dosage form. The additive or polymer may have at least one common or shared chemical and/or physical and/or biological property of the many chemical/physical/biological properties of a polymer selected from a carbomer such as Carbopol 934P. At least one “shared property” of the polymer is preferably having side chains or groups that are ionizable. Such groups include, for example, carboxylic acid groups or other ionizable moieties such as sulfate or phosphate precursors (e.g. C—OH groups substituted with —SO₃H or —PO₃H size chains or variables). The relative percentage by weight on a dry basis of carboxylic acid groups or other ionizable or neutralizable groups in the polymer preferably ranges from 40-80%. Other shared properties include, but are not limited to, hydrophilicity and/or swellability and/or gelability and/or viscosity (i.e., aqueous viscosity in mPa s). Carbopol 934 P has an aqueous viscosity in a 0.5% wt/vol solution of 29,400-39,400 mPa s. Shared properties can be chemical, physical or biological. Shared biological properties include, for example, sharing the delivery properties of a Carbopol polymer across a cell membrane or tissue by transcellular means or by paracellular means through, for example, duodenal tissue or other epithelial tissue. The additive or polymer may have more than one shared property with a carbomer. The percentage of additive or agent in the formulation relative to the active ingredient can range from about 0.1% to about 80% or more on a wt/wt percentage basis. The preferred weight ratio of additive to active, when a polymeric additive is present, is 1:1 or greater (e.g. 1:1; 1.5:1; 2:1; 2.5:1; 3:1 etc). The polymeric additive is not a required additive. The compound of formula Compound 1a may also be delivered without such an additive and in a suitable vehicle with a pharmaceutically acceptable excipient or filler such as lactose or without any fillers in, for example, an appropriate saline or aqueous solution for aerosol delivery or for oral delivery.

The pharmaceutically acceptable polymer may be selected from a natural polymer such as an alginate or mixtures or alginic acid and complex salts of alginic acid which may be water soluble or water insoluble. Natural alginic acids and complexes thereof are generally described in, for example, U.S. Pat. No. 4,842,866. Alginate gums or natural polymers or gums similar to alginate gums (e.g. carrageenan gums, xanthan gums, tragacanth gums, locust bean gums, guar gums or any other complex polymers derived from plant, microbial or other natural sources and which are pharmaceutically acceptable) may be utilized in the formulation of the invention.

The pharmaceutically acceptable synthetic polymer may be selected from a hydrophobic or hydrophilic polymer. The polymer may be water soluble, slightly water soluble or water insoluble. The water soluble hydrophilic polymers may be selected from the group consisting of polyvinylpyrrolidone, hydroxypropyl cellulose, hydroxypropylmethyl cellulose, methyl cellulose, vinyl acetate/crotonic acid copolymers, methyacrylic acid polymers and copolymers, maleic anhydride/methyl vinyl ether copolymers and derivatives and mixtures thereof. The polymers may be low viscosity polymers with viscosity ranging from about 50 cps to about 200 cps and can include commercially available polymers such as Methocel™ K100 LV and similar polymers from the Dow Chemical Company. The water soluble hydrophilic polymers may also be selected from, for example, sodium carboxymethyl cellulose or other similar anionic water soluble polymers. These polymers can include polyhydroxyalkyl methacrylates, anionic or cationic hydrogels, polyvinyl alcohol or high molecular weight polyethylene oxides such as those described in various patents including, for example, U.S. Pat. No. 4,837,111.

The pharmaceutically acceptable synthetic polymer may also be selected from hydrophilic water-insoluble polymers. These are polymers that can readily absorb water, become hydrated and/or swell. These polymers can be selected from carbomers which include various Carbopol homopolymer polymers such as carboxyvinyl polymers and carboxy polymethylene or polyacrylic acid copolymers. The preferred polymers are Carbopol polymers of acrylic acid cross-linked with polyalkenyl ethers or divinyl glycol. These polymers swell and can also form gels under various conditions. The preferrend Carbopol polymers include Carbopol 934P NF; Carbopol 974P NF; Carbopol 971P NF and Carbopol 71G. Other ionic polymers suitable for use in the formulation include sodium alginate, calcium carboxymethyl cellulose, sodium carboxymethyl cellulose or methyacrylic acid, acrylic acid ethyl ester copolymer. The Carbopol polymers are used in oral suspensions but are also used in dry formulations in, for example, capsules which contain or comprise a disaccacharide, a Carbopol polymer and a filler such as lactose. Thus, the present invention also relates to oral suspensions or capsules or other solid dosage forms comprising a compound of formula I or II as described above and an additive selected from a polymer that swells when in contact with water or ionizes or is neutralizable or has a chemical group that facilitate the delivery or transport of the active ingredient to the site of action. The capsules or tablets may be coated with further excipients or polymers including enteric polymers. The coating materials may be selected from, for example, enteric coatings such as cellulose acetate phthalate, cellulose acetate trimelliate, hydroxypropylmethyl cellulose phthalate, copolymers of methacrylic acid and ethyl acrylate (e.g. Eudragit L30D), hydroxypropylmethyl cellulose acetate succinate or polyvinyl acetate phthalate. The preferred coatings are highly stable in the acidic environment of the stomach but break down in the more basic environment of the small intestine.

Hydrophobic polymers or additives may be selected from, for example, ethyl cellulose, polymeric methacrylic acid esters, cellulose acetate butyrate, poly(ethylele-co-vinyl-acetate), hydroxyethyl cellulose, and methacrylate polymers selected from the Eudragit polymers. Additional hydrophobic additives may be selected from waxes or fatty acid esters. It is preferred that these hydrophobic polymers swell or contain additional polymers to form blends or mixtures that swell or ionize when exposed to water or “gel”. Additional “agents” that enhance the delivery of the hypersulfated disachamides include, but are not limited to, polyanionic salts (such as polyanionic salts of glutamic acid or aspartic acid); glycosaminoglycans such as hyaluronic acid; modified amino acids; modified amino acid derivatives; alkali swellable rheology modifiers; polyoxyethylene glycols; fatty acid esters; chitosan (high and low molecular weight versions as described in U.S. Pat. No. 7,291,598 and poly-glutamic acid and nanoparticles thereof; bile salts and acids thereof alone and in combination with surfactants and optional solubilizers; phospholipid polyvalent cations; phospholipase C inhibitors; unilamellar vesicles; sulphated chitinous polymers; permeabilizing reagents selected from iminodiacetic acid, nitriloacetic acid, ethylene diaminomono acetic acid, ethylene diamino diacetic acid, ethylene diamino tetraacetic acid, sodium taurodihydro fusidate, sodium caprate, sodium glycocholate, cholylsarcosine, isopropyl myristate, partially hydrolyzed triglycerides, fatty acid sugar derivatives and oleic acid derivatives; and biodegradable polymers such as poly(lactide co glycolide). Such agents are disclosed in the following publications or patents and which are herein incorporated by reference in their entirety: U.S. Pat. Nos. 5,498,410; 5,827,512; 5,908,637; 5,990,096; 6,458,383; 6,461,643; 6,635,702; 6,855,332; 7,291,598; 7,329,638; US20010024658; US20020037316; US20020115641; US20030180348; US20040038870; US20040086550; US20040096504; US20070287683 and US20090082321. These agents may be added instead of or in addition to the previously described natural or synthetic polymers.

The formulations of the invention can be delivered to the patient or other organism by any suitable known means. The percentages of the additive and type of additive added to the formulation relative to the active ingredient and other excipients Will be based upon the type of formulation desired. For example, in an oral suspension formulation to be delivered to a patient or organism in need of treatment thereof, the vehicle can be an oral liquid or oral capsule. The preferred formulations are an oral capsule or inhalation formulation.

The compositions or compounds of the invention further comprise pharmaceutically acceptable excipients and/or fillers and extenders such as lactose or other sugars including but not limited to glucose, sucrose, mannitol etc. and lubricants such as magnesium stearate, talc, calcium stearate, solid polyethylene glycols, sodium lauryl sulfate and mixtures thereof. The amount of filler or lubricant or other known pharmaceutically acceptable additive will vary based upon the type of formulation and the manner the formulation is processed or made.

The compositions of the invention can be delivered or administered orally in the form of tablets, capsules or suspensions. The tablets or capsules can be prepared by means known in the art and contain a therapeutically effective amount of a hypersulfated disaccharide of formula I or II according to the invention in addition to the recited delivery agent including, for example, a polymeric additive. Tablets and pills or other suitable formulations can be prepared with enteric coatings and other release controlling coatings. Coatings can be added to afford light protection or swallowability. The capsules and tablets or suspensions can include additives which improve the taste of the medicine.

Liquid dosage forms for oral administration may include pharmaceutically acceptable emulsions, solutions, suspensions, syrups and elixers containing inert diluents such as water as well as the compounds of formula I and salts thereof and the additives selected from a pharmaceutically acceptable polymers. Such formulations may additionally include adjuvants including wetting agents, emulsifying and suspending agents, and sweetening, flavoring and perfuming agents. Inhalation delivery formulations will include, in addition to the active ingredient, suitable delivery vehicles or propellants or the active ingredient may be in the form of a dry powder. Such delivery Means and systems are well known to those of ordinary skill in the art. The active ingredient may also be delivered via nebulizers in a suitable delivery system. Such nebulizable formulations are also well known in the art. Breath activated inhalers may also be utilized to deliver the active ingredient.

The compounds of formula I and II form, as stated above, pharmaceutically acceptable salts. The metal salts include for example salts having Na, K, Ca, Ng or Ba or Zn, Cu, Zr, Ti, Bi, Mn or Os or salts formed by reacting the compounds of formula I or II with an organic base such as an amino acid or with any amine. The preferred salt is a sodium salt.

Thus, the preferred formulations of the invention includes the compound designated as Compound 1a (octasulfated sucrose sodium salt) and which further and optionally include a pharmaceutical excipient or a delivery agent selected from, for example, an additive selected from an ionic swellable hydrophilic insoluble polymer such as Carbopol 934 P. The preferred formulations are administered in the form of capsules or via inhalation using, for example, an aerosol formulation. The aerosol formulation may be delivered via inhaler or a nebulizer or other suitable inhalation means. Nasal sprays may also be used to deliver Compound 1a.

These formulations are useful in treating a number of inflammatory diseases and conditions. Types of respiratory diseases or conditions contemplated herein include allergic rhinitis which is characterized by seasonal or perennial sneezing, rhinorrhea, nasal congestion, and often conjunctivitis and pharyngitis; acute rhinitis, characterized by oedema of the nasal mucosa, nasal discharge and mucosa. Pulmonary diseases, such as intrinsic or extrinsic bronchial asthma, any inflammatory lung disease, acute and chronic bronchitis, pulmonary inflammatory reactions secondary to chronic bronchitis, chronic obstructive lung disease, pulmonary fibrosis, Goodpasture's syndrome as well as any lung disease or condition in which white blood cells may play a role including idiopathic pulmonary fibrosis and any other autoimmune lung disorders are treatable with the formulation of the invention.

Ear, nose and throat disorders such as acute external otitis, furunculosis and otomycosis of the external ear are treatable by the formulations of the invention. Other conditions include respiratory diseases such as traumatic and infectious myringitis, acute Eustachian salpingitis, acute serous otitis media and acute and chronic sinusitis.

Formulations of the invention are useful in treating pulmonary inflammation. The term “pulmonary inflammation” encompasses any inflammatory lung disease, acute chronic bronchitis, chronic obstructive lung disease, pulmonary fibrosis, Goodpasture's syndrome, and any pulmonary condition in which white blood cells may play a role including but not limited to idiopathic pulmonary fibrosis and any other autoimmune lung disease.

Formulations of the invention are useful in treating asthma and asthma related pathologies. The term “asthma” means a condition of allergic origin, the symptoms of which include continuous or paroxysmal labored breathing accompanied by wheezing, a sense of constriction in the chest, and, often, coughing or gasping. The term “asthma related pathologies” means a condition whose symptoms are predominantly inflammatory in nature with associated bronchospasm. Both asthma and an asthma related pathology are characterized by symptoms which include a narrowing of the airways, varying over short periods of time either spontaneously or as a result of a treatment, due in varying degrees to contraction (spasm) of smooth muscle, edema of the mucosa, and mucus in the lumen of the bronchi and bronchioles. Generally these symptoms are triggered by local release of spasmogens and vasoconstrictive substances (e.g. histamine or certain leukotrienes or prostaglandins) in the course of an allergic response. Non-limiting examples of asthma related pathologies include non-asthmatic conditions characterized by airway hyperresponsiveness (e.g. chronic bronchitis, emphysema and cystic fibrosis). The most prominent characteristic of asthma is bronchospasm, or narrowing of the airways: asthmatic patients have prominent contraction of smooth muscles of large and small airways, increased mucous production, and increased inflammation. The inflammatory response in asthma is typical for tissues covered by mucosa and is characterized by vasodilation, plasma exudation, recruitment of inflammatory cells such as neutrophils, monocytes, macrophages, lymphocytes, and eosinophils to the sites of inflammation and the release of inflammatory mediators by resident tissue cells (mast cells) or by migrating inflammatory cells (J.C. Hogg, “Pathology of Asthma,” Asthma and Inflammatory Disease, P. O'Byren (ed.), Marcel Dekker, Inc., New York, N.Y. 1990, pp. 1-13).

Asthma may be triggered by multiple or a variety of causes such as in response to allergens, secondary exposure to infective agents, industrial or occupational exposures, ingestion of chemicals, exercise and/or vasculitis (Hargreave et al., J. Allergy Clinical Immunol. 83:1013-1026, 1986). As discussed herein, there may be two phases to an allergic asthma attack-an early phase and a late phase which follows 4-6 hours after bronchial stimulation (Harrison's Principles of Internal Medicine 14^{th} Edl, Fauci et al. (eds), McGraw Hill, New York, N.Y. 1998, pp. 1419-1426). The early phase which typically resolves spontaneously, includes the immediate inflammatory response including the response caused by the release of cellular mediators from mast cells. The late phase reactions develop over a period of hours and are characterized histologically by an early influx of polymorphonuclear leukocytes and fibrin deposits followed by infiltration of eosinophils. A certain percentage of patients are “dual responders” and develop an early acute and a late phase response. In dual responders, the acute phase is followed 4-14 hours later by a secondary increase in airway resistance (“late phase response” or LPR or “late airway response” or LAR). Late responders and dual responders are of particular clinical importance because, in combination with the airway inflammation, late phase responses lead to a prolonged airway hyperreactivity (AHR), asthmatic exacerbations, or hyperresponsiveness, worsening of symptoms, and generally a more severe form of clinical asthma that may last from days to months in some subjects, requiring aggressive therapy. Pharmacological studies in allergic animals have demonstrated that not only the bronchoconstrictor response but also the inflammatory cell influx and the mediator release pattern in dual responders is quite different from acute responders.

An increase in bronchial hyperreactivity (AHR), the hallmark of a more severe form of asthma, can be induced by both antigenic and non-antigenic stimuli. Last phase response, allergen-induced asthma and persistent hyperresponsiveness have been associated with the recruitment of leukocytes, and particularly, eosinophils, to inflamed lung tissue (W. M. Abraham et al., Am. Rev. Respir. Dis. 138: 1565-1567, 1988). Eosinophils release several inflammatory mediators including 15-HETE, leukotriene C4, PAF, cationic proteins and eosinophil peroxidase.

Moreover, the formulations of the invention are also useful in treating late phase reactions and inflammatory response in extra pulmonary sites such as allergic dermatitis, inflammatory bowel disease; rheumatoid arthritis and other collagen vascular diseases, glomerulonephritis, inflammatory skin diseases and conditions; and sarcoidosis.

As used herein, the term “treating or alleviating the symptoms” means reducing, preventing and/or reversing the symptoms of the individual to which a formulation of the invention has been administered as compared to the symptoms of the individual or an individual which is untreated. Hence, a formulation of the invention that treats or alleviates the symptoms of asthma or an asthma related pathology reduces, prevents, and/or reverses the early phase asthmatic response to antigen challenge in a dual responder individual, more preferably reduces, prevents and/or reverses the late phase asthmatic response to antigen challenge in a dual responder individual, and more preferably reduces, prevents and/or reverses both the early phase and late phase responses to antigen challenge in a dual responder individual. This “treatment” or “alleviation” is preferably a significant percentage as shown in the animal models presented herein for the recited formulations and with respect to LAR and AHR data.

The terms “antigen” and “allergen” are used interchangeably to describe those substances such as dust or pollen that can induce an allergic reaction and/or induce an asthmatic episode or asthmatic symptoms in an individual suffering from such condition. Thus an individual is “challenged” when an allergen or antigen is present in a sufficient amount to trigger an asthmatic response in such individual.

It is also understood that the formulations of the invention are useful in treating any disease or condition affected by late phase reactions (LPR's). The airways are merely a prototype of organs or tissues affected by such LPR's. It has been established in the medical literature that the last phase bronchoconstriction and AHR observed in dual responder asthmatic patients is not an isolated phenomenon restricted to asthmatic or even pulmonary patients. Thus, the present formulation is useful in treating any disease or condition affected by LPR's including cutaneous, nasal, ocular and systemic manisfestations of LPR's in addition to pulmonary associated LPR's. Clinical diseases (whether of the skin, lung, nose, eye or other organs) recognized to involve allergic mechanisms have a histologic inflammatory component which follows the immediate allergic or hypersensitivity reaction that occurs on antigen challenge. This sequence of response appears to be connected to mast cell mediators and propogated by other resident cells within target organs or by cells recruited into the sites of mast cell or basophilic degranulation. Thus, the present formulation is useful in treating inflammatory bowel disease, rheumatoid arthritis, glomerulonephritis and inflammatory skin disease. The present invention therefore relates to a method of treating a patient or organism in need of treatment thereof and who/which is suffering from a disease or condition characterized by late phase allergic reactions, including e.g, and without limitation, pulmonary, nasal, cutaneous, ocular and systemic LPR's, and/or which is characterized by inflammatory reactions through the administration, by any known means, of a formulation comprising a compound of formula I or II and a delivery agent such as, for example, a polymeric additive to said patient or organism.

The term “inflammatory condition” means a disease, condition or symptom selected from the group consisting of pulmonary inflammation such as asthma and/or asthma related pathologies; pneumonia, tuberculosis, rheumatoid arthritis, allergic reactions which impact the pulmonary system, early and late phase responses in asthma and asthma related pathologies, diseases of the small and large airways of the lung, bronchospasm, inflammation, increased mucus production, conditions which involve vasodilation, plasma exudation, recruitment of inflammatory cells such as neutrophils, monocytes, macrophages, lymphocytes and eosinophils and/or release of inflammatory mediators by resident tissue cells (mast cells); conditions or symptoms which are caused by allergens, secondary responses to infections, industrial or occupational exposures, ingestion of certain chemicals or foods, drugs, exercise or vasculitis; conditions or symptoms which involve acute airway inflammation, prolonged airway hyperreactivity, increases in bronchial hyperreactivity, asthmatic exacerbations, hyperresponsiveness; conditions or symptoms which involve the release of inflammatory mediators such as 15-HETE, leukotriene C4, PAF, cationic proteins or eosinophil peroxidases; conditions or symptoms which relate to cutaneous, nasal, ocular or systemic manifestations of late phase allergic responses; clinical diseases of the skin, lung, nose, eye or throat or other organs and which involve allergic mechanisms having an histologic inflammatory component upon antigen challenge; allergic rhinitis, respiratory diseases characterized by seasonal or perennial sneezing; rhinorrhea, conjunctivitis, pharyngitis, intrinsic or extrinsic bronchial asthma, any inflammatory lung disease, acute and chronic bronchitis, pulmonary inflammatory reactions secondary to acute chronic bronchitis, chronic obstructive lung disease (COPD), pulmonary fibrosis, Goodpasture's syndrome, any pulmonary condition in which white blood cells play a role including but not limited to idiopathic pulmonary fibrosis and any other autoimmune lung disease; ear, nose and throat disorders such as acute external otitis, furunculosis and otomycosis of the external ear; respiratory diseases such as traumatic and infectious myringitis; acute eustachian salpingitis, acute serous otitis media, acute and chronic sinitis; extrapulmonary conditions selected from any late-phase reactions and inflammatory response such as allergic rhinitis; allergic dermatitis; allergic conjunctivitis; extrapulmonary diseases where inflammation occurs and/or an inflammatory response plays a major role including inflammatory bowel disease; rheumatoid arthritis and other collagen vascular diseases; glomerulonephritis; inflammatory skin diseases and sarcoidosis and cardiovascular inflammation, especially inflammation associated with coronary atherosclerosis, as described below.

The present formulation comprising Compound 1a may also be utilized to treat inflammatory conditions associated with cardiovascular disease. It is known that there are serious side effects associated with traditional anti-inflammatory agents such as glucocorticoid steroids and cyclophosphamide making them inappropriate choices for treatment of atherosclerotic inflammation. On the other hand, the polysulfated disaccharide formulations of the invention have the advantage of having few side effects along with anti-inflammatory properties. It has clearly been postulated that atherosclerotic lesions are due to or have many properties associated with chronic inflammation including the presence of macrophages, lymphocytes and denditric cells which accumulate at specific loci to cause and/or acerbate lesions. L. K. Curtiss, N. Engl. J. Med. 360; 11 1144-1146 (2009). The present formulation is thus useful for the treatment of arteriosclerotic disorders in patients having such disorders or conditions and is further useful in the treatment or prevention of restenosis after invasive vascular surgery or after an organ transplant. The formulation suitable for cardiovascular treatment can be administered by any known means including by interal or parenteral administration. The present invention comprises a method of treating cardiovascular inflammation comprising administration of a composition comprising a compound of formula I wherein R₁-R₈ are as defined herein and pharmaceutically acceptable salts thereof and an optional pharmaceutically acceptable excipient or delivery agent to a patient in need of treatment thereof. The present invention further includes combinations of a compound of formula I with R₁-R₈ as defined herein and a cardiovascular drug selected from an HMGCoA reductase inhibitor or other cardiovascular drug or drugs used to treat cardiovascular disease. The “combination” may be in the form of a single dosage form having at least two active ingredients wherein one of the active ingredients is a hypersulfated disaccharide of the invention and the other active ingredient is selected from an HMGCoA reductase inhibitor such as lovastatin, simvastatin, atorvastatin or rosavastatin calcium. The combination includes a formulation of the invention comprising a compound of formula I or II wherein R₁-R₈ is as defined herein along with a pharmaceutically acceptable excipient or additive such as a polymer or delivery agent and a second active ingredient selected from an HMGCoA reductase inhibitor.

The formulations of the invention have been found to be effective in animal studies which are predictive of utility in humans as well as other animals. The animal studies demonstrate that the formulations are useful in (a) preventing antigen-induced bronchoconstrictor response and bronchial hyperactivity (also referred to as airway-hyperresponsiveness (AHR)) and (b) in ameliorating AHR subsequent to antigen challenge in treated animals. Pulmonary airflow resistance was measured by taking allergic sheep previously verified as dual bronchoconstrictor responders to Ascaris suum antigen. The sheep were intubated with a cuffed nasotracheal tube and pulmonary airflow resistance (R_{L}) was measured by the esophageal balloon catheter technique, while thoracic gas volume was measured by body plethysmography. Data were expressed as specific R_{L} (SR_{L}, defined as R_{L} times thoracic gas volume (V_{tg})). Airway responsiveness was determined by first securing cumulative dose response curves to inhaled carbachol (a constrictor agonist) by measuring SR_{L} before and after inhalation of buffered saline and after each administration of 10 breaths of increasing concentrations of carbachol (0.25, 0.5, 1.0, 2.0 and 4.0% wt/vol solution). Airway responsiveness was measured by determining the cumulative provocation dose (PD₄₀₀) of carbachol (in breath units) that increased SR_{L} to 400% above baseline. One breath unit was defined as one breath of 1% carbachol solution.

As appropriate, and according to the prescribed method of administration, the formulations of the invention may be administered prior to, at the same time, or after the organism or patient has been exposed to an antigen and in relation to the particular disease or condition being treated. Doses of the active ingredient (the hypersulfated sucrose of formula I or II) may range from less than 1 mg to 1,000 mgs per day. Suitable doses may also range from 0.001 mgs/kg/day to 100 mgs/kg/day or higher per treated organism. The preferred dose ranges from 0.1 mgs/kg/day to 1 mg/kg/day. One of ordinary skill in the art can modify the dose per patient or per patient groups to treat the diseases or conditions referenced herein. Capsules, tablets or suspensions may be formulated for once or twice a day administration and at doses including 5 mgs, 10 mgs, 15 mgs, 20 mgs, 25 mgs, 30 mgs, 35 mgs, 40 mgs, 45 mgs, 50 mgs, 100 mgs, and 200 mgs of active ingredient. The capsules or tablets or oral suspensions further include at least 0.1 percent (on a wt/wt basis) an additive which is selected from a polymer (natural or synthetic) or other/additional agent that enhances delivery of the active drug as recited herein. The formulations may also be inhalation formulations and using doses that are in the range of those suggested above. For example, an aerosol formulation may comprise a compound of formula I or II in a range of 0.1 to 100 mgs per delivery along with a suitable aerosol or vehicle.

The formulations of the invention may be administered alone or in combination with other suitable medications or active ingredients and depending upon the particular disease or condition being treated. In a preferred embodiment, the formulations or compounds of the invention are administered in the morning or evening. Thus, the present invention comprises a method of treating a disease or condition associated with antigen exposure and which involves an early and late phase response comprising administering to an organism in need thereof a therapeutically effective amount of a compound of formula I or II with R₁-R₈ as defined herein (i.e., with at least two sulfate groups) and a delivery enhancing agent wherein the formulation is administered in the morning or evening. The invention further comprises a method of treating a disease or condition associated with antigen exposure and which involves an early and late phase response comprising administering to an organism in need thereof a therapeutically effective amount of a compound of formula I or II with R₁-R₈ as defined herein and a pharmaceutically acceptable excipient or a natural or synthetic polymer or other/additional delivery enhancing agent to form a formulation and wherein said formulation is administered to the organism in the morning or evening. The additional active ingredients that may be administered in the form of combination therapy or in the form of a single dosage unit having at least two active ingredients wherein the first active is a compound of formula I or II with R₁-R₈ as defined herein and a second active selected from any drug or medicament which is used as front line therapy to treat asthma or an asthma related disorder or condition or other inflammatory condition as recited herein. Such medicaments include anti-inflammatories, leukotriene antagonists or modifiers, anticholinergic drugs, mast cell stabilizers, corticosteroids, immunomodulators, beta-adrenergic agonists (short acting and long acting), methyl xanthines, and other general classes or specific drugs used to treat such disorders including, but not limited to, montelukast sodium; albuterol; levoalbuterol; salmeterol; formoterol, fluticasone propionate; budesonide; ceterizine; loratadine; desloratadine; theophylline, ipratropium, cromolyn, nedocromil, beclomethasone, flunisolide, mometasone, triaminoclone, prednisoline, prednisone, zafirlukast, zileuton or omalziunab.

The following examples are intended to further illustrate certain embodiments of the invention and are non-limiting.

### Example 1

### Preparation of Octasulfated Sucrose

A stirred solution of sucrose (5 gm) and pyridine-sulfur trioxide complex (14.05 gm) in anhydrous pyridine (50 mL) and DMF (10 mL) or pure DMF (60 mL) was heated to 55-65° C. and stirred for 6 to 18 hours. The reaction mixture was cooled to room temperature (25° C.) and the solvent removed under reduced pressure. The semi-solid residue obtained was suspended in a 5% water/methanol solution (100 mL) and stirred for 20-30 minutes at room temperature. The suspension was filtered, the filter cake re-suspended in aqueous MeOH solution and stirred for 20-30 minutes at room temperature. The suspension was filtered, the filtrates were combined and concentrated under reduced pressure. The solid residue obtained was dissolved in purified water (50 mL) and the solution pH adjusted to 6.8 (±0.1) with sodium hydroxide solution. Activated charcoal (10 g) was added to the neutralized solution, the suspension stirred vigorously for 20 minutes and filtered through diatomaceous earth (Celite). The decolorized solution was freeze dried to afford the crude supersulfated material as a solid. Size exclusion chromatography of the solid on a 1.5m×90 cm column containing a BioRad P4 (or P2) BioGel (10 mL) and eluting with 0.2 M NH₄HCO₃ provided the ammonium salt of the supersulfated sucrose (2.3 gm) after freeze-drying of the appropriate fractions. The ammonimum salt of the supersulfated sucrose was exchanged for the sodium salt by passing an aqueous solution of the ammonium salt through a column containing Amberlite IR120PLUS Cation Exchange Resin (150 gm). The filtrate from the ion exchange column may again be decolorized with activated carbon and then freeze dried to afford the product (Compound 1a) as a white to off white solid (2.3 gm).

### Example 2

### Pulmonary Evaluation of An Animal Model (Sheep)

To illustrate the effectiveness of the formulations according to the invention to treat and alleviate allergen related diseases and conditions, including but not limited to the specific diseases and conditions recited herein, sheep were assessed in multiple experiments which compared various formulations containing no added polymer or additive to animals which were provided formulations comprising a compound of formula I (as compound 1a) along with an optional additive selected from a polymer. To measure pulmonary airflow resistance, the sheep were intubated with a cuffed nasotracheal tube and pulmonary airflow resistance (R_{L}) was measured by the esophageal balloon catheter technique, while thoracic gas volume was measured by body plethysmography. These methods are accepted and well known methods found in the literature. Data were expressed as specific R_{L} (SR_{L} defined as R_{L}× thoracic gas volume (V_{g}).

To assess airway responsiveness, cumulative dose response curves to inhaled carbachol were performed by measuring SR_{L} before and after inhalation of buffered saline and after each administration of 10 breaths of increasing concentrations of carbachol (0.25, 0.5, 1.0, 2.0, and 4.0% wt/vol solution). Airway responsiveness was measured by determining the cumulative provocation dose (PD₄₀₀) of carbachol (in breath units) that increased SR_{L} to 400% above baseline. One breath unit was defined as one breath of 1% carbachol solution.

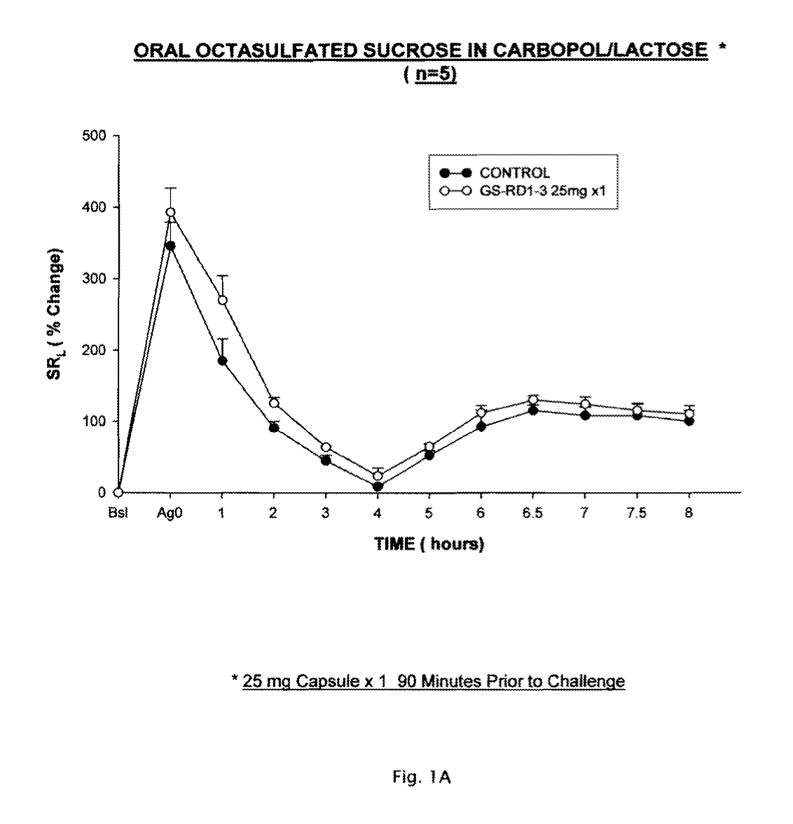

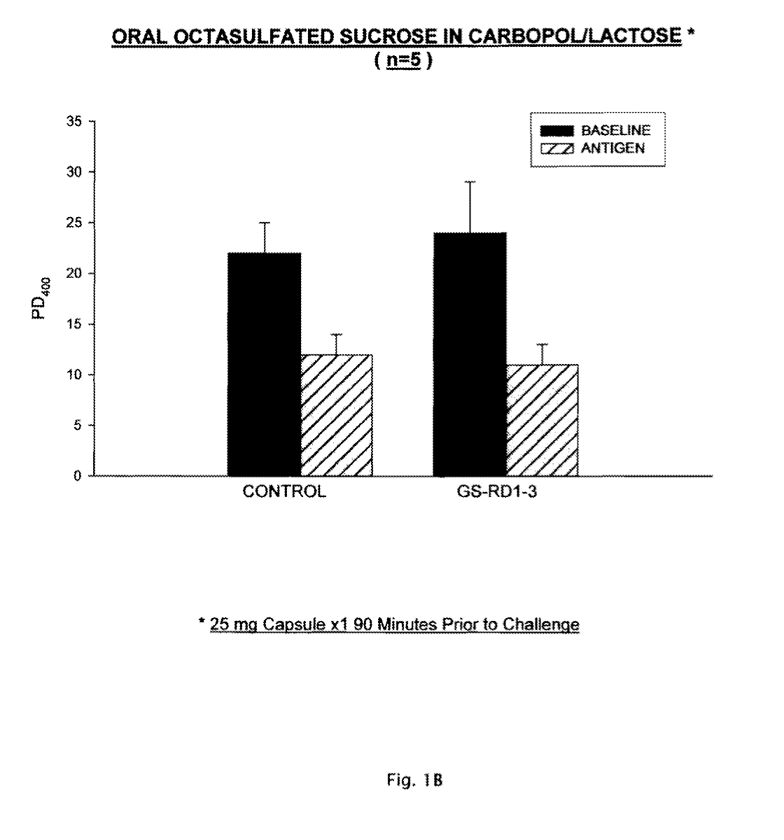

For airway studies, each animal's baseline airway responsiveness (PD₄₀₀)) was determined and then, on different experimental days, the test sheep underwent airway challenge with Ascaris suum antigen. SR_{L} was measured to establish baseline, then measured again immediately after antigen challenge and hourly for an eight hour period and then a post challenge PD₄₀₀ was measured 15-24 hours after antigen challenge. In each of the Figures presented herein, FIGS. 1A, 2A, 3A etc. present day two data measured on an hourly basis for the eight hour period and contain control data (closed circles) and drug treatment data (open circles). The drug treatment experiments were conducted on the same animals used in the control studies but after a period of several weeks following the day 3 PD₄₀₀ measurements. FIGS. 1B, 2B, 3B etc. contain the day one baseline PD₄₀₀ data and day three PD₄₀₀ data following antigen challenge in control or drug treated animals.

Data were expressed or may be expressed as (a) mean+/−SE % change of SR_{L} and (b) PD₄₀₀ in breath units. Data were also expressed as (c) % protection of Early Airway Response (EAR, for 0-4 hours) and Late Airway Response (LAR, for 4-8 hours), as estimated by area under the curve for EAR and LAR respectively. And (d) AHR %

$$protection = 100 - ( \frac{Baseline {PD}_{400} - {drug}_{antigen} {PD}_{400}}{Baseline {PD}_{400} - {Control}_{antigen} {PD}_{400}} \times 100 )$$

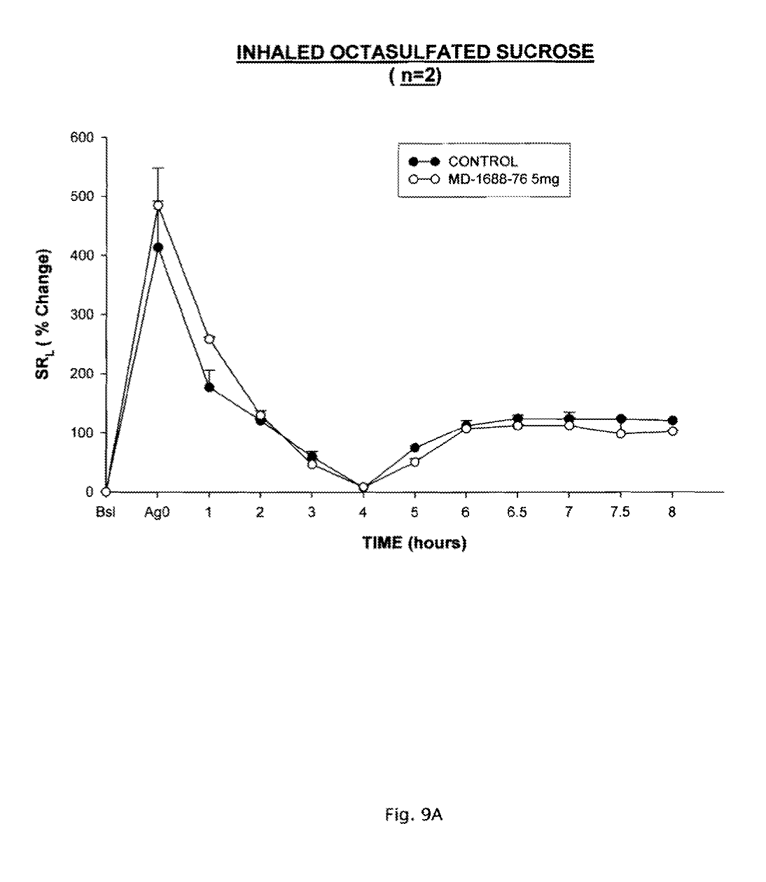

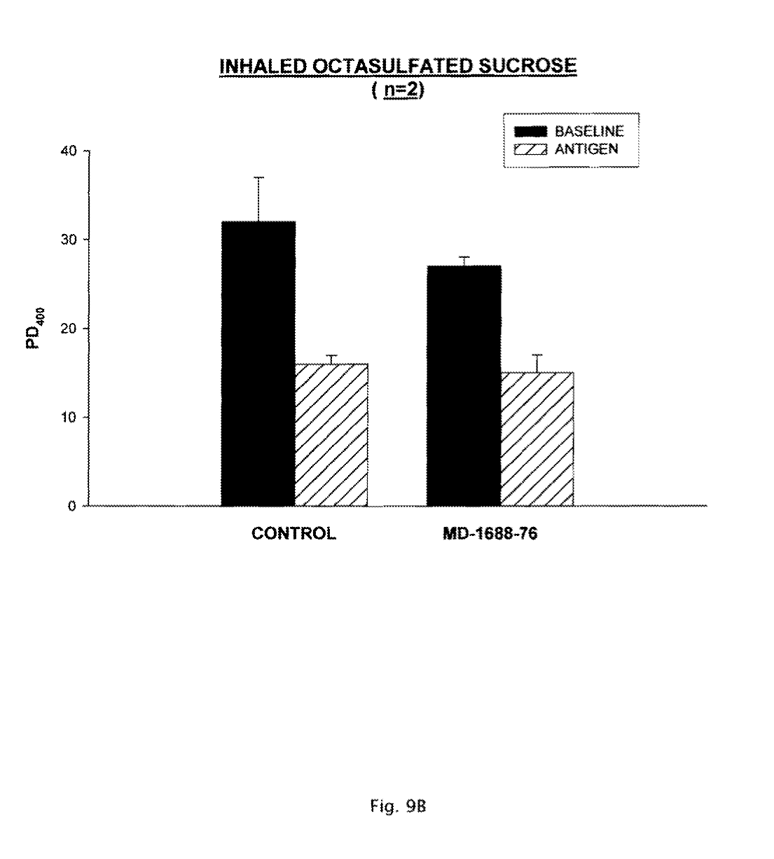

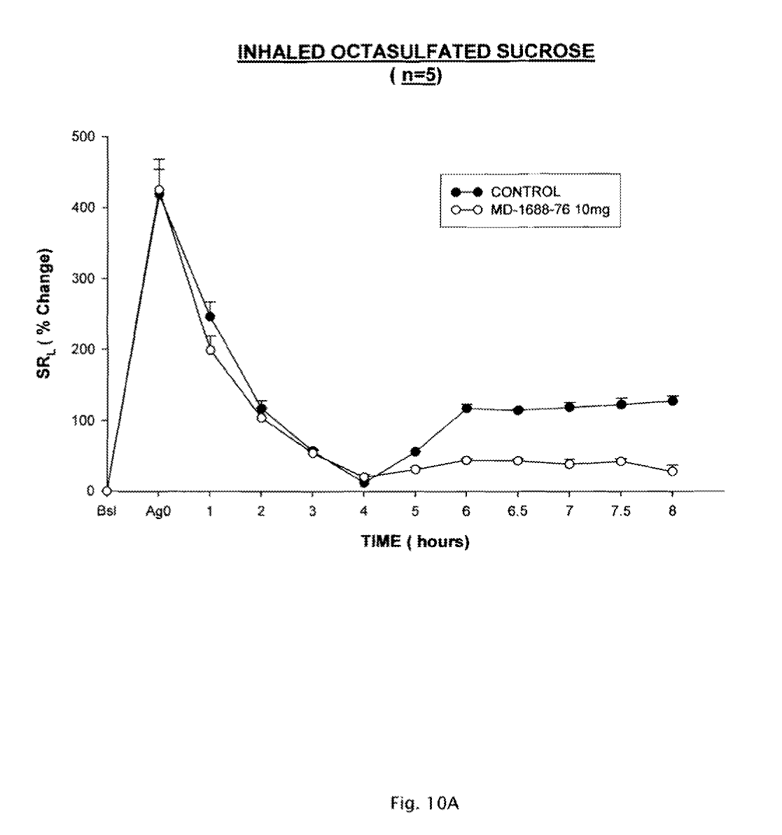

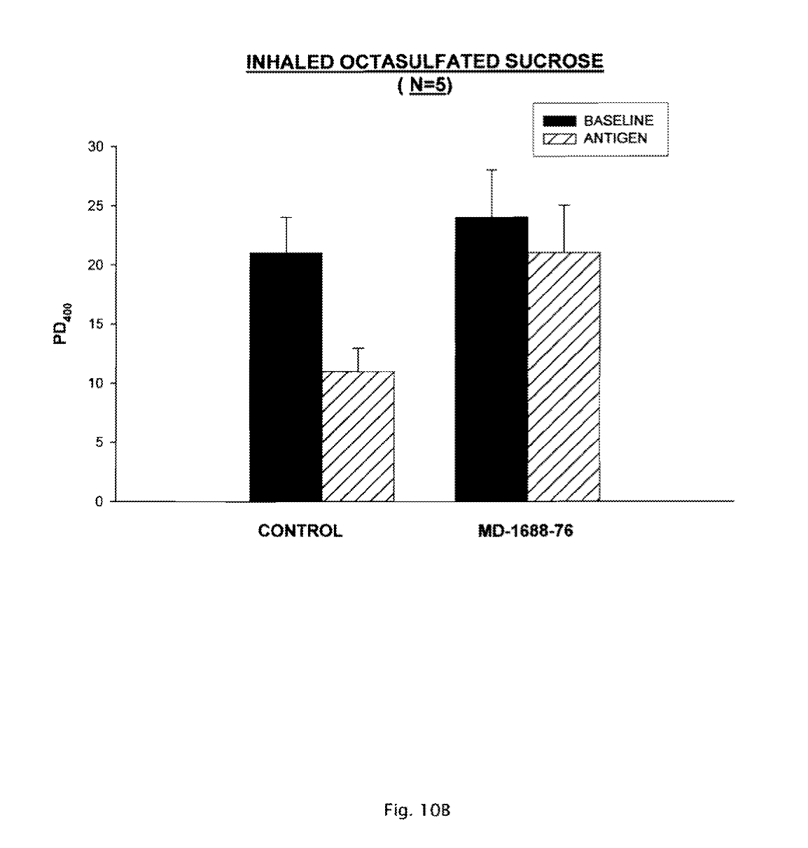

As an example, in FIG. 9B, Baseline PD₄₀₀-drug_{antigen}PD₄₀₀ was 30-15; Baseline PD₄₀₀-Control_{antigen}PD₄₀₀ was 27-13. 15/14×100=105. 100-105=0% protection in AHR for the 5 mg dose. In contrast, the % protection for the 10 mg dose shown in FIG. 10B was 24-22 and 20-11 which gave 2/9×100=23. 100-23=77% protection.

In the studies presented in FIGS. 1A-7B, the data shows the % change in SR_{L} and PD₄₀₀ in breath units for Control antigen response studies and for Drug-Treated antigen response studies. In the drug treated animals, oral capsule doses were given with or without a polymeric additive and at different dosage strengths of Compound 1a. FIG. 1A shows the % change over time in SR_{L} in animals relative to control at an oral dosage of 25 mg (one capsule) of compound 1a in a Carbopol/lactose formulation(GS-RD1-3) when given ninety minutes before antigen challenge. As can be seen in FIG. 1A, there is no significant effect on EAR (0-4 hr) between control and drug treatment, and there is no significant positive effect on LAR (4-8 hr) in the period following exposure to antigen due to drug treatment (LAR % protection=0%). As can be seen in FIG. 1B, the oral dosage of a 25 mg capsule also had no effect on airway hyperresponsiveness (AHR % protection=<0%).

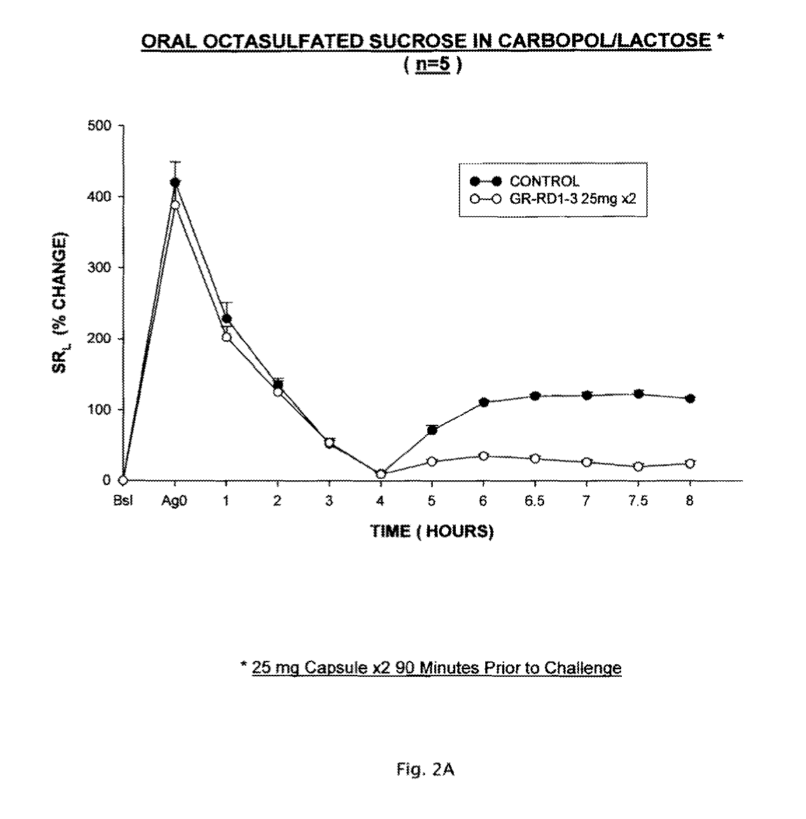

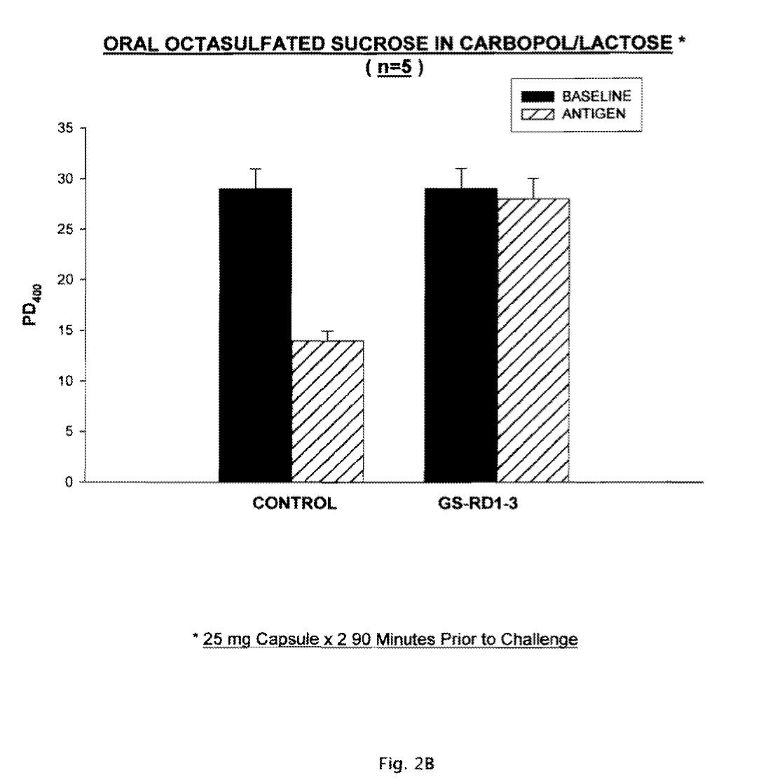

FIG. 2A shows the % change over time in SR_{L} in animals relative to control at an oral dosage of 50 mgs (25 mg capsule×2 of compound 1a (GS-RD1-3) when given ninety minutes before antigen challenge. As can be seen in FIG. 2A, there is no effect on EAR between control and drug treatment, but there is a more significant positive effect on LAR following exposure to antigen due to drug treatment (LAR % protection=86%). As can be seen in FIG. 2B, the oral dosage of 25 mgs×2 also had a more significant positive effect on airway hyperresponsiveness (AHR % protection=88%) versus the dose given using one 25 mg capsule.

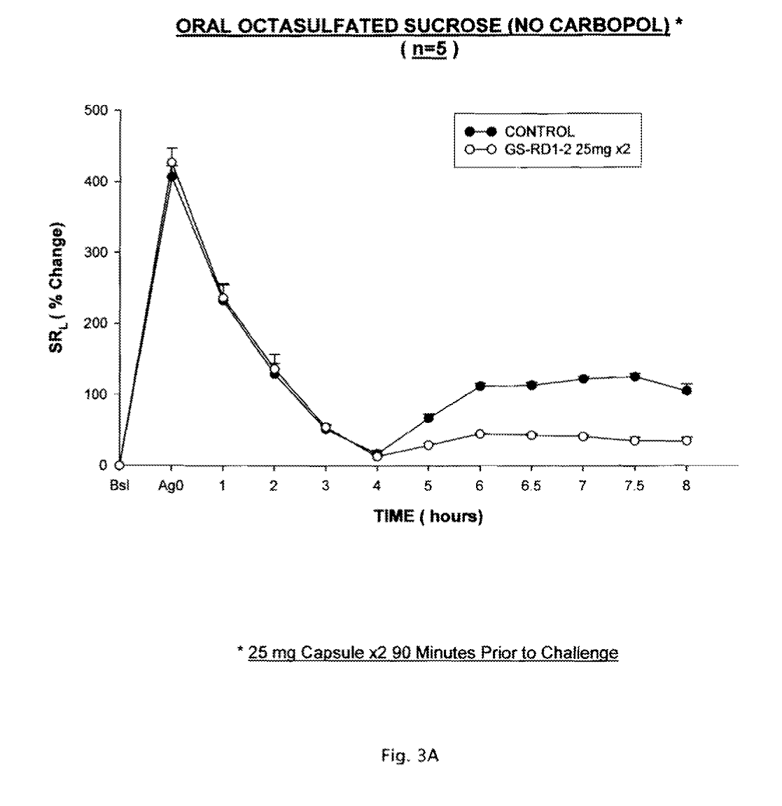

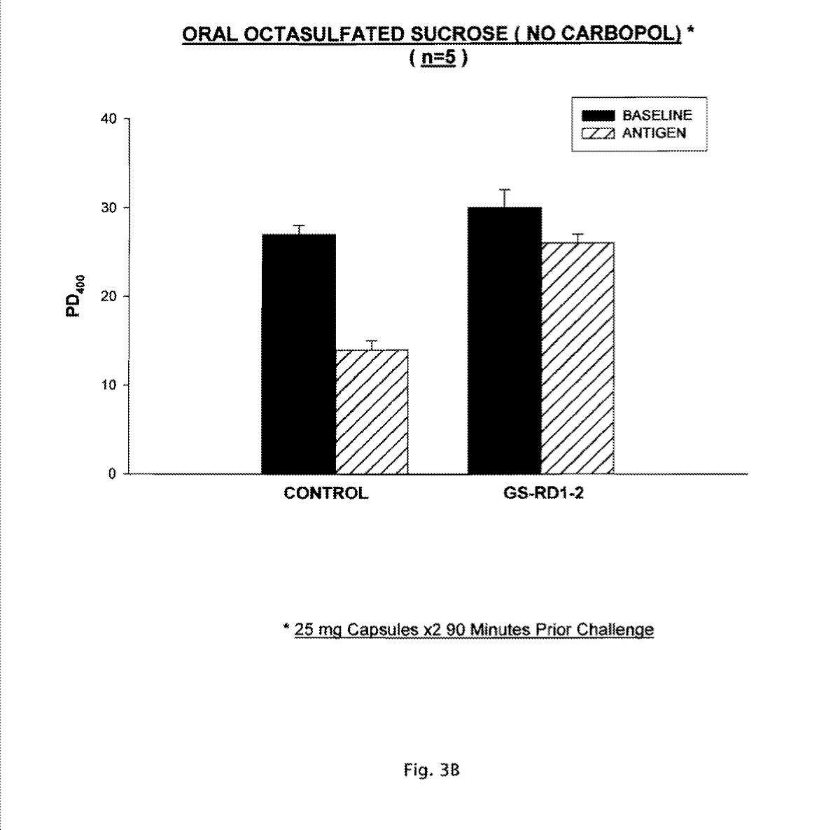

FIG. 3A shows the % change over time in SR_{L} in animals relative to control at an oral dosage of 50 mgs (2×25 mg capsules) of compound 1a in a formulation that did not have Carbopol (GS-RD1-2) administered as two 25 mg capsules. Antigen challenge occurred 90 minutes after dosing. As can be seen in FIG. 3A, there was no effect on EAR between control and drug treatment, but there was a significant positive effect on LAR following exposure to antigen due to drug treatment (LAR % protection=70%). As can be seen in FIG. 3B, the oral dosage of two 25 mg doses also had marked effect on airway hyperresponsiveness (AHR % protection=74%) indicating effective drug treatment.

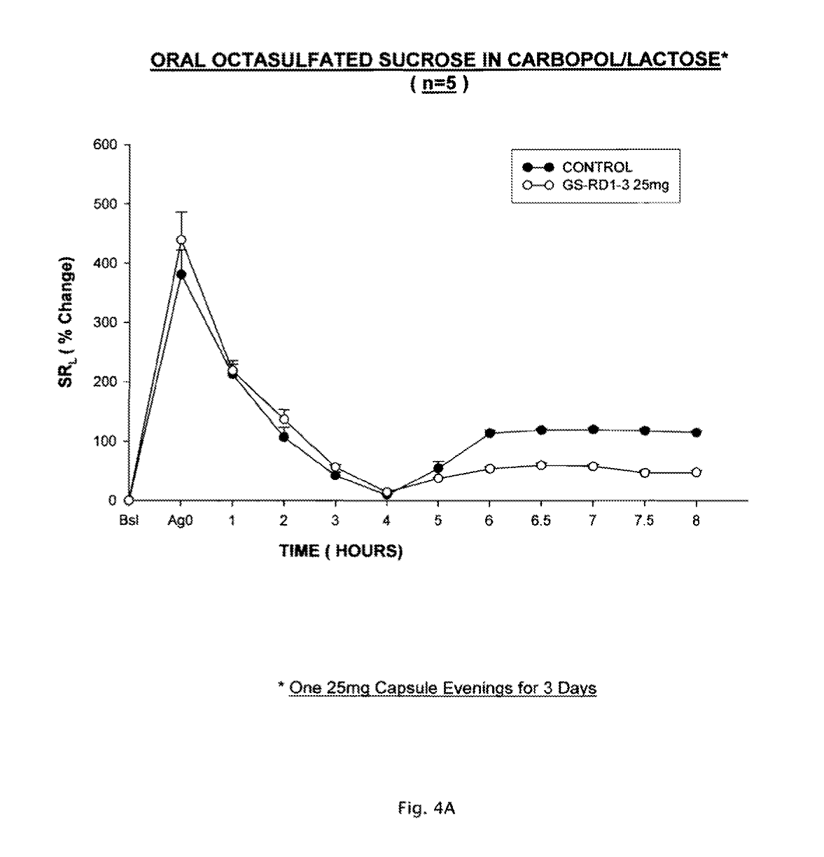

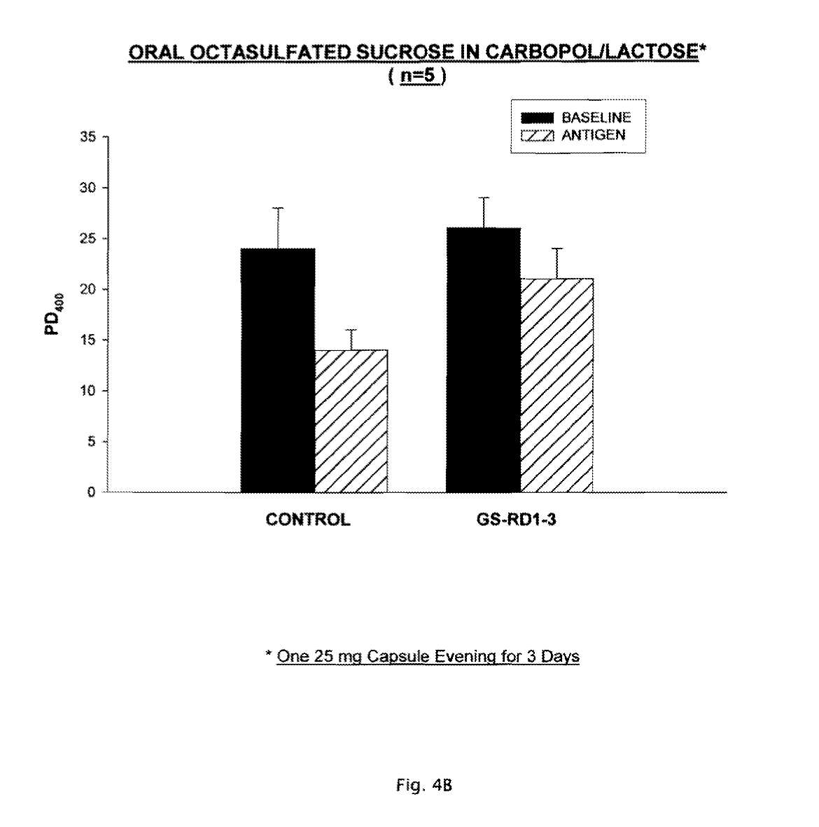

FIG. 4A shows the % change over time in SR_{L} in animals relative to control at an oral dosage of one 25 mg capsule given for three days in the evening of compound 1a in a Carbopol containing formulation (GS-RD1-3). Antigen challenge occurred 15 hours after the last P.M. dose. As can be seen in FIG. 4A, there was no effect on EAR between control and drug treatment but there was a significant positive effect on LAR which was due to drug treatment (LAR % protection=49%). As can be seen in FIG. 4B, the oral dosage of one 25 mg capsule administered in the above manner also had a positive effect on airway hyperresponsiveness (AHR % protection=53%).

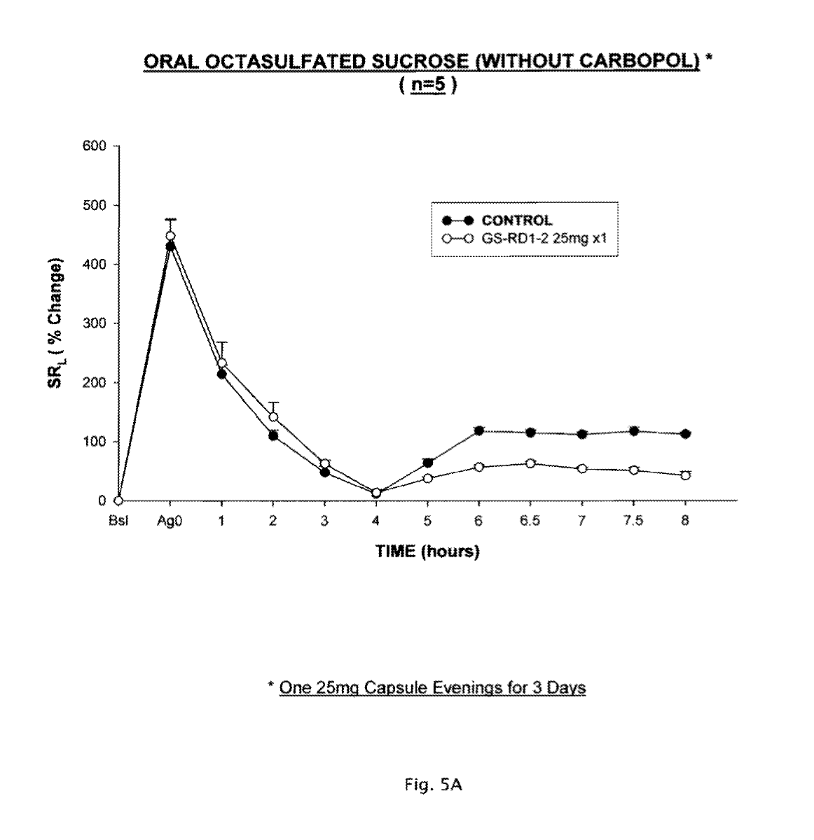

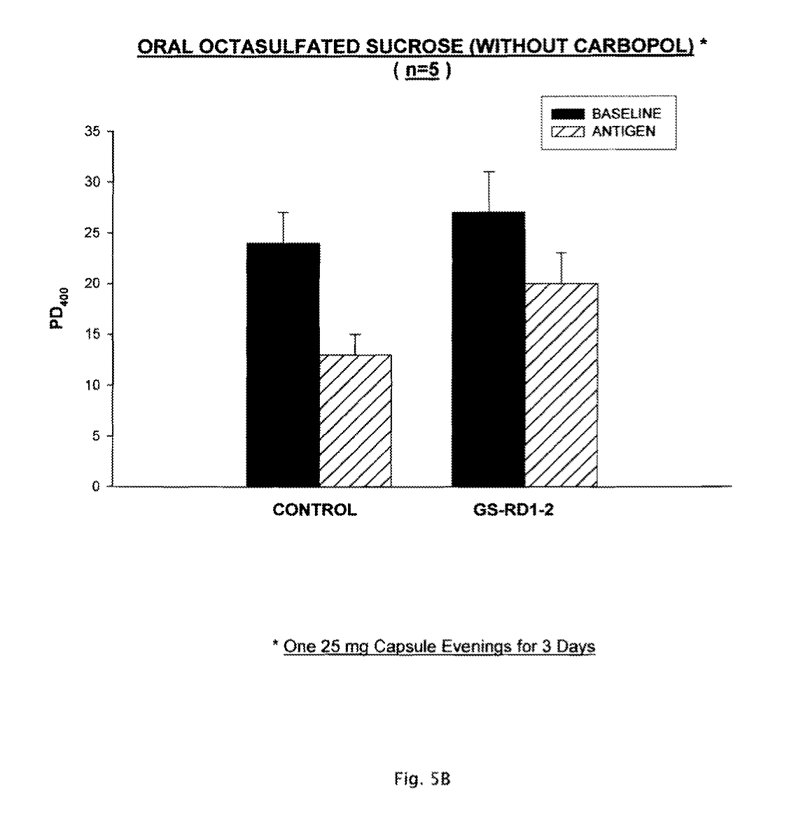

FIG. 5A shows the % change over time in SR_{L} in animals relative to control at an oral dosage of one 25 mg capsule of compound 1a in a Carbopol free formulation (GS-RD1-2) administered in the evening (P.M.) over a three day period before antigen exposure: Antigen challenge occurred 15 hours following the last evening dose. As can be seen in FIG. 5A, there is no effect on EAR between control and drug treatment and there is an effect on LAR following exposure to antigen due to drug treatment (LAR % protection=49%). As can be seen in FIG. 5B, the oral dosage of 25 mgs once a day for three days also had a positive effect on airway hyperresponsiveness (AHR % protection=40%).

The weight of sheep used in the studies was between 30-40 kg (avg. wt. 35 kg). Thus, for comparison purposes, a 20 mg dose given once a day is administered at an average dose of about 0.6 mg/kg/day-e.g. 20 mgs/35 kg/day.

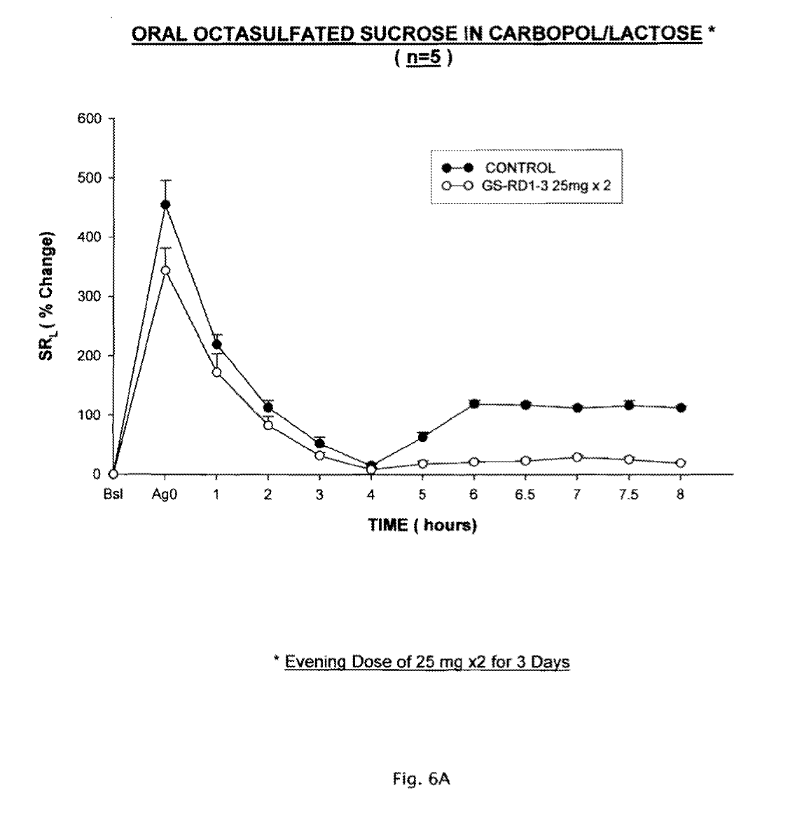

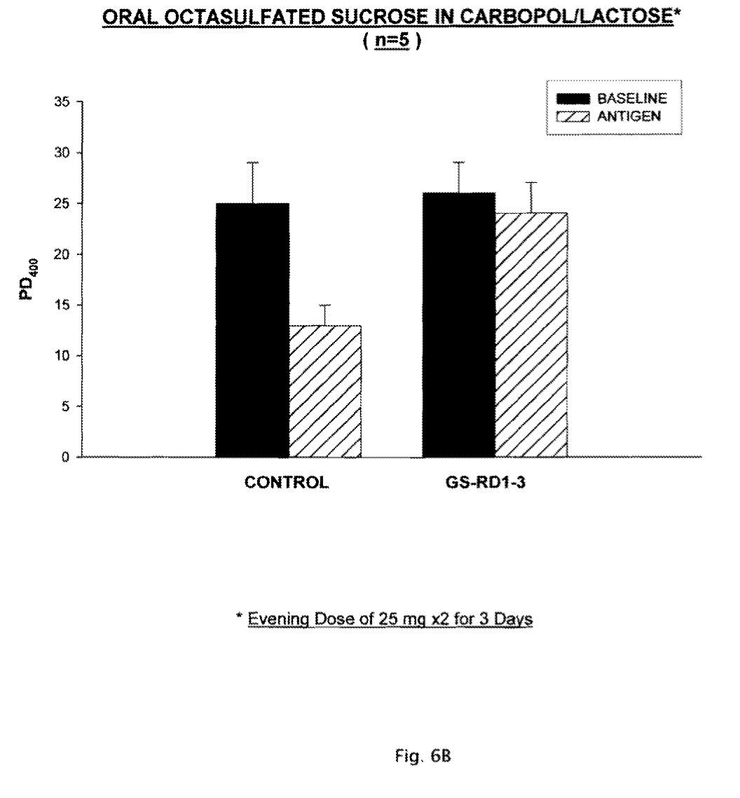

FIG. 6A shows the % change over time in SR_{L} in animals relative to control at an oral dosage of 50 mgs of compound 1a (2×25 mg enteric coated capsules having 50 mgs Carbopol 934 P with lactose filler) (formulation GS-RD1-3) (1:2 wt/wt) administered over a three day period at night (P.M.) and with antigen challenge 24 hours following the last 2×25 mg dose. As can be seen in FIG. 6A, there is a positive effect on EAR between control and drug treatment (EAR protection=25%) and there is a significant positive effect for LAR following exposure to antigen due to drug treatment (LAR % protection=78%). As can be seen in FIG. 6B, the oral dosage of 25 mgs×3 days given at night also had a positive effect on airway hyperresponsiveness (AHR % protection=91%).

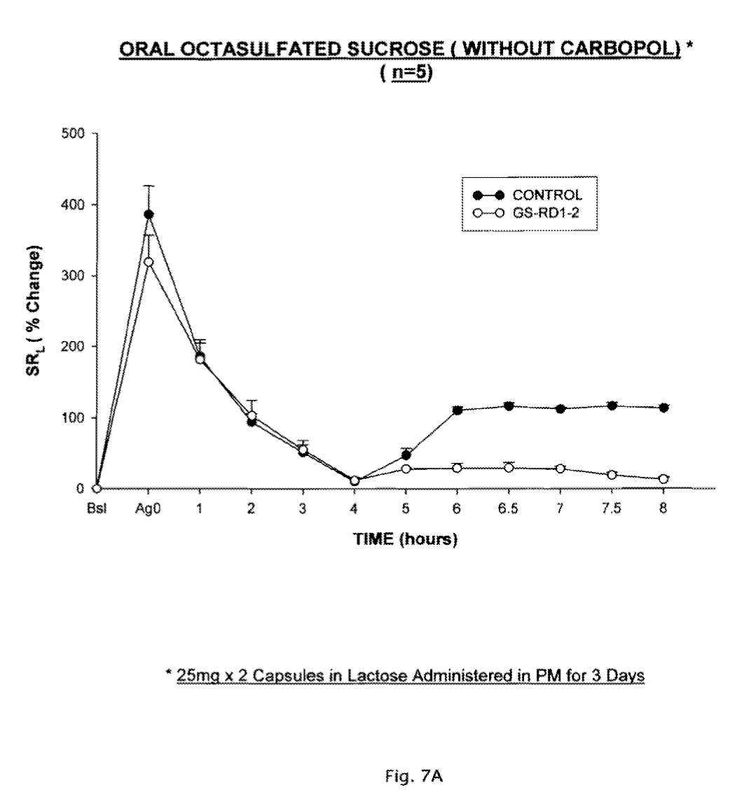

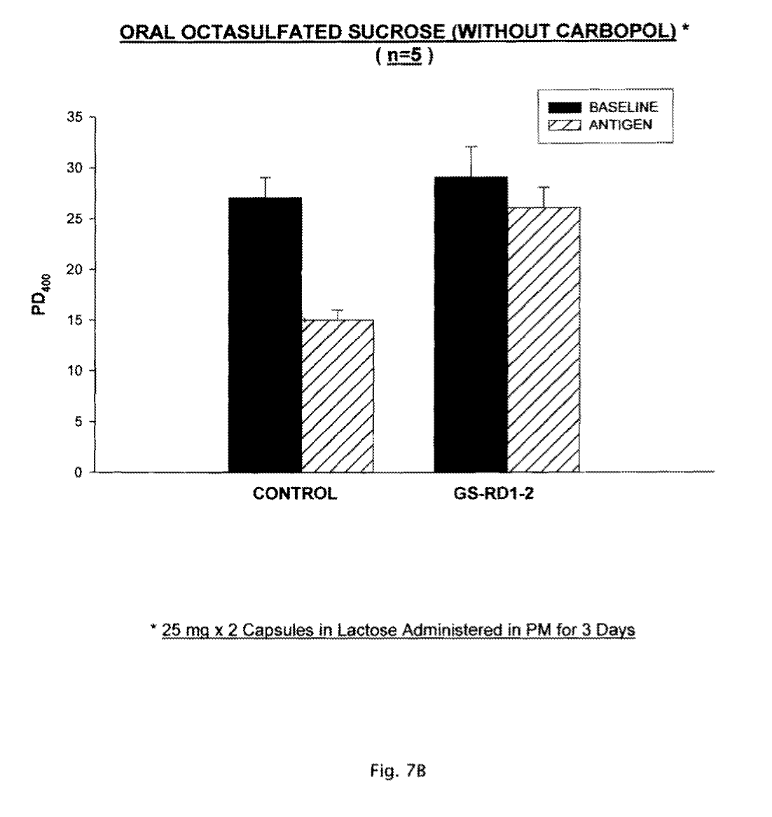

FIG. 7A shows the % change over time in SR_{L} in animals relative to control at an oral dosage of 50 mgs of compound 1a and no Carbopol (formulation GS-RD1-2) administered as two 25 mg enteric coated capsules over a three day period at night. Antigen challenge occurred 15 hours following the last 25 mg treatment. As can be seen in FIG. 7A, there is no significant effect on EAR between control and drug treatment but there is a significant positive on LAR following exposure to antigen due to drug treatment (LAR % protection=72%). As can be seen in FIG. 7B, the oral dosage of 25 mgs times 3 days at night also had a positive effect on airway hyperresponsiveness (AHR % protection=74%).

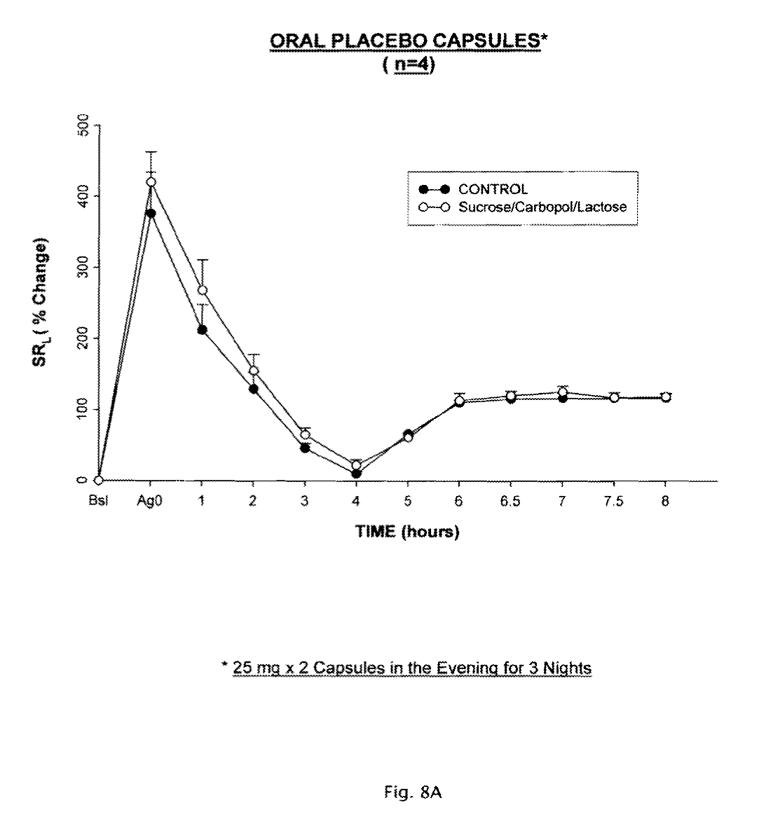

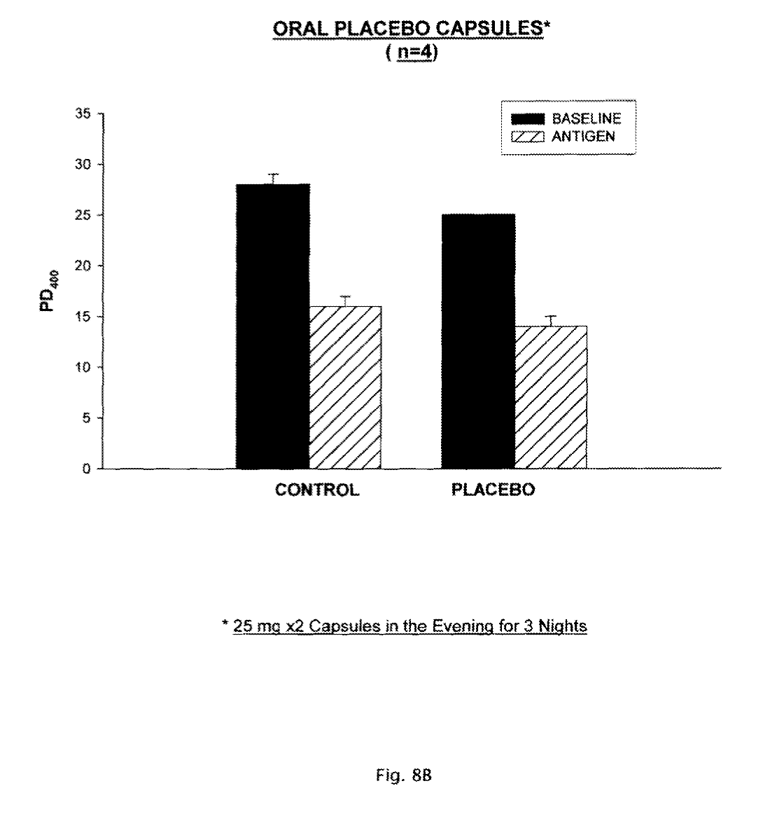

FIG. 8A shows the % change over time in SR_{L} in animals relative to control at an oral dosage of 50 mgs sucrose and 100 mgs Carbopol 934 P (formulation MD1599-72) administered as two 25 mg enteric coated capsules over a three day period at night. Antigen challenge occurred 15 hours following the last 50 mg sucrose treatment. As can be seen in FIG. 8A, there is no significant positive effect on EAR between control and drug treatment and there is no significant positive effect on LAR following exposure to antigen due to sucrose treatment (LAR % protection=0%). As can be seen in FIG. 8B, the oral dosage of 50 mgs sucrose time 3 days at night also had no positive effect on airway hyperresponsiveness (AHR % protection=0%).

FIG. 9A shows the % change over time in SR_{L} in animals relative to control at an inhaled dosage of 5 mgs of Compound 1a in an aerosol formulation (MD-1688-76 5 mg). Antigen challenge occurred thirty minutes following inhalation. As can be seen in FIG. 9A, there is no positive effect on EAR between control and placebo treatment and there is no positive effect in LAR following exposure to antigen due to placebo treatment (LAR % protection=0). As can be seen in FIG. 9B, the inhaled dosage of 5 mgs also had no positive effect on airway hyperresponsiveness (AHR % protection=0).

FIG. 10A shows the % change over time in SR_{L} in animals relative to control at an inhaled dosage of 10 mgs of compound 1a (formulation MD1688-76 10 mg). Antigen challenge occurred thirty minutes following drug treatment. As can be seen in FIG. 10A, there is no positive effect in EAR between control and drug treatment and there is a significant positive effect in LAR following exposure to antigen due to drug treatment (LAR % protection=60%). As can be seen in FIG. 10B, the inhaled dosage of 10 mgs also had a positive effect on airway hyperresponsiveness (AHR % protection=71%).

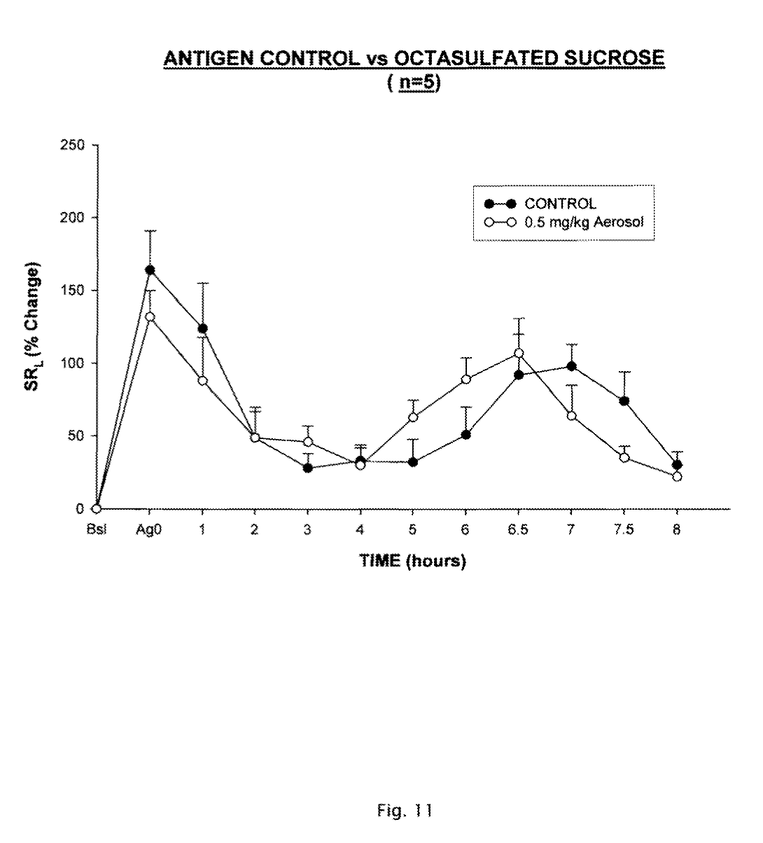

FIG. 11 shows the % change over time in SR_{L} in animals relative to control in an aerosol formulation dosage of 0.5 mgs/kg of the aluminum salt of octasulfated sucrose. Antigen challenge occurred thirty minutes following treatment. As can be seen in FIG. 11A, there is no significant effect on EAR between control and drug treatment and there is essentially no positive effect in LAR following exposure to antigen due to drug treatment (LAR % protection=0%).

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize or be able to ascertain using no more than routine experimentation, numerous embodiments of the claimed invention which may not have been expressly described. Such embodiments are within the scope of the invention.

1. A pharmaceutical formulation suitable for treating a pulmonary disease or condition comprising a compound of formula I and pharmaceutically acceptable salts thereof 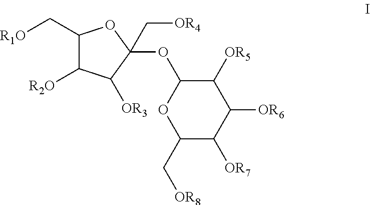
wherein R₁-R₈ are independently selected from the group consisting of H, SO₃H or PO₃H and provided that at least two of R₁-R₈ is selected from SO₃H or PO₃H; and an additive selected from the group consisting of a pharmaceutically acceptable excipient or a delivery agent.

2. The formulation according to claim 1 wherein at least three of R₁-R₈ is selected from SO₃H or PO₃H.

3. The formulation according to claim 1 wherein at least four of R₁-R₈ is selected from SO₃H or PO₃H.

4. The formulation according to claim 1 wherein at least five of R₁-R₈ is selected from SO₃H or PO₃H.

5. The formulation according to claim 1 wherein R₁-R₈ is selected from SO₃H.

6. A pharmaceutical formulation comprising a compound of formula II and pharmaceutically acceptable salts thereof 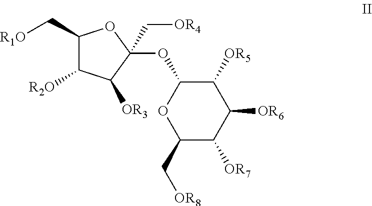
wherein R₁-R₈ are selected from —SO₃H and an additive selected from the group consisting of a pharmaceutically acceptable excipient or a polymer.

7. A pharmaceutical formulation according to claim 6 comprising a compound of formula II and pharmaceutically acceptable salts thereof wherein the pharmaceutically acceptable salt is a sodium salt.

8. The formulation according to claim 7 wherein the pharmaceutically acceptable polymer is a hydrophilic polymer.

9. The formulation according to claim 8 wherein the hydrophilic polymer is selected from a crosslinked polymer of acrylic acid.

10. The formulation according to claim 9 wherein the cross-linked polymer of acrylic acid is selected from Carbopol 934P.

11. A method of treating or alleviating an inflammatory condition in a mammal in need of treatment thereof comprising administration of
(i) a pharmaceutically effective amount of a formulation comprising a compound of formula I or II 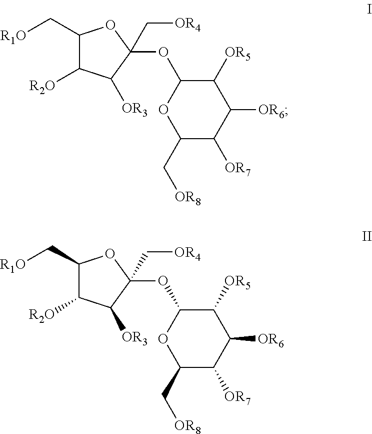
and pharmaceutically acceptable salts thereof wherein R₁-R₈ are independently selected from —SO₃H or —PO_{S}H and, optionally,
(ii) an additive selected from a pharmaceutically acceptable excipient or polymer.

12. The method according to claim 11 wherein the compound is selected from a compound of formula II and the pharmaceutically acceptable salt is a sodium salt.

13. The method according to claim 11 wherein R₁-R₈ is selected from SO₃H.

14. The method according to claim 13 wherein the optional polymer is selected from a water insoluble, hydrophilic swellable polymer.

15. The method according to claim 14 wherein the water insoluble, hydrophilic swellable polymer is selected from an acrylic acid polymer.

16. The method according to claim 11 wherein the inflammatory condition is selected from pulmonary inflammation such as asthma and/or asthma related pathologies; pneumonia, tuberculosis, rheumatoid arthritis, allergic reactions which impact the pulmonary system, early and late phase responses in asthma and asthma related pathologies, diseases of the small and large airways of the lung, bronchospasm, inflammation, increased mucus production, conditions which involve vasodilation, plasma exudation, recruitment of inflammatory cells such as neutrophils, monocytes, macrophages, lymphocytes and eosinophils and/or release of inflammatory mediators by resident tissue cells (mast cells); conditions or symptoms which are caused by allergens, secondary responses to infections, industrial or occupational exposures, ingestion of certain chemicals or foods, drugs, exercise or vasculitis; conditions or symptoms which involve acute airway inflammation, prolonged airway hyperreactivity, increases in bronchial hyperreactivity, asthmatic exacerbations, hyperresponsiveness; conditions or symptoms which involve the release of inflammatory mediators such as 15-HETE, leukotriene C4, PAF, cationic proteins or eosinophil peroxidases; conditions or symptoms which relate to cutaneous, nasal, ocular or systemic manifestations of late phase allergic responses; clinical diseases of the skin, lung, nose, eye or throat or other organs and which involve allergic mechanisms having an histologic inflammatory component upon antigen challenge; allergic rhinitis, respiratory diseases characterized by seasonal or perennial sneezing; rhinorrhea, conjunctivitis, pharyngitis, intrinsic or extrinsic asthma bronchiale, any inflammatory lung disease, acute or chronic bronchitis, pulmonary inflammatory reactions secondary to acute chronic bronchitis, chronic obstructive lung disease (COPD), pulmonary fibrosis, Goodpasture's syndrome, any pulmonary condition in which white blood cells play a role including but not limited to idiopathic pulmonary fibrosis and any other autoimmune lung disease; ear, nose and throat disorders such as acute external otitis, furunculosis and otomycosis of the external ear; respiratory diseases such as traumatic and infectious myringitis, acute eustachian salpingitis, acute serous otitis media, acute and chronic sinitis; extrapulmonary conditions selected from any late-phase reactions and inflammatory response such as allergic rhinitis; allergic dermatitis; allergic conjunctivitis; extrapulmonary diseases where inflammation occurs and/or an inflammatory response plays a major role including inflammatory bowel disease; rheumatoid arthritis and other collagen vascular diseases; glomerulonephritis and inflammatory skin diseases and sarcoidosis.

17. The method according to claim 16 wherein the inflammatory condition is selected from pulmonary inflammation.

18. The method according to claim 16 wherein the mammal in need of treatment thereof is human.

19. An oral dosage form comprising
(i) a compound of formula I or a pharmaceutically acceptable salt thereof 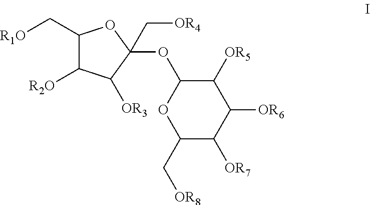
wherein R₁-R₈ are independently selected from SO₃H or PO_{S}H and, optionally,
(ii) an additive selected from the group consisting of a pharmaceutically acceptable excipient or a polymer.

20. An inhalation dosage form comprising
(I) A compound of formula I or a pharmaceutically acceptable salt thereof 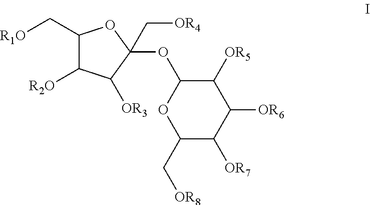
wherein R₁-R₈ are independently selected from SO₃H or PO₃H and, optionally,
(ii) an additive selected from the group consisting of a pharmaceutically acceptable excipient.

21. An inhalation dosage form comprising
(i) A compound of formula II or a pharmaceutically acceptable salt thereof 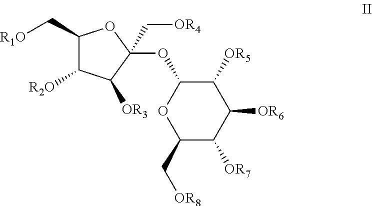
wherein R₁-R₈ are independently selected from SO₃H or PO₃H and, optionally,
(ii) an additive selected from the group consisting of a pharmaceutically acceptable excipient.

22. The inhalation dosage form according to claim 21 wherein R₁-R₈ are selected from —SO₃H and the compound is in the form of the sodium salt.
